(19) United States
(12) Patent Application Publication
(10) Pub. No.: US 2004/0106975 A1
(43) Pub. Date: Jun. 3, 2004

(54) Rail stent

(75) Inventors: Kenneth S. Solovay, Weston, FL (US); Thomas P. Jacobs, Fort Lauderdale, FL (US)

Correspondence Address: GMP COMPANIES, INC., ONE EAST BROWARD BLVD., SUITE 1701, FORT LAUDERDALE, FL, 33301, US

(73) Assignee: GMP/Cardiac Care, Inc., Fort Lauderdale, FL

(21) Appl. No.: 10/713,873

(22) Filed: Nov. 14, 2003

Related U.S. Application Data
(63) Continuation-in-part of application No. 10/100,986, filed on Mar. 20, 2002.
(60) Provisional application No. 60/276,913, filed on Mar. 20, 2001.
(60) Provisional application No. 60/426,366, filed on Nov. 15, 2002.

(51) Int. Cl. A61F 2/06

(52) U.S. Cl. 623/1.11

(57) ABSTRACT

A stent with a plurality of support elements that are deployable within a body for supporting a vessel or other body structure. The stent includes first and second terminal ends and a length extending between the terminal ends. Support rails extend between the terminal ends and through the support members in a direction parallel to the longitudinal axis of the stent. The support elements can include openings for receiving the rails. The rails can include curved end sections that aid in deployment of the stent into a vessel.

### CROSS-REFERENCES TO RELATED APPLICATIONS

This application is a continuation-in-part of U.S. patent application Ser. No. 10/100,986 filed on Mar. 20, 2002, which claims the benefit of U.S. Provisional Patent Application No. 60/276,913 filed on Mar. 20, 2001, and this application also claims the benefit of U.S. Provisional Patent Application No. 60/426,366, filed on Nov. 15, 2002. The full disclosure of each of these applications is incorporated herein by reference.

### FIELD OF THE INVENTION

The present invention relates to a stent for use in supporting vascular tissue, and particularly, to a stent having improved longitudinal structural flexibility.

### BACKGROUND OF THE INVENTION

It is generally known to insert a resiliently expandable stent into a blood vessel to provide radial vascular support (hoop support) within the vessel in the treatment of atherosclerotic stenosis. For example, it is generally known to open a blocked cardiac blood vessel by known methods (e.g., balloon angioplasty or laser ablation) and to keep that blood vessel open using such a stent. These stents are generally formed of a biocompatible material, such as stainless steel, and have slots or holes cut therein so a balloon can expand the stent after being deployed into the blood vessel

However, a conventional stent structure tends to be longitudinally inflexible (i.e., along a length of the stent), and therefore tends to be resistant to transverse deformation. As a result, the conventional stent tends to straighten a blood vessel into which it is inserted because it resists conforming to the shape of a curved blood vessel path. Currently, there is some discussion in the art regarding a relationship between this tendency to straighten a blood vessel and the onset of restensosis (i.e., blood vessel reclosure).

Conventional, longitudinally inflexible stents are disclosed in, for example, U.S. Pat. No. 6,113,628 to Borghi and U.S. Pat. No. 5,653,727 to Wiktor. The stents discussed in these patents are not capable of achieving the longitudinal flexibility needed to prevent restenosis. These stents include circumferential vascular support elements (sometimes referred to as “hoops”) that are securely spaced from each other and from the ends of the stent so that they do not experience relative axial movement. The spacing between adjacent support elements is maintained by rigid connections or bridge elements (sometimes referred to in the art as “bridges”) that extend between adjacent support elements and/or a rigid connection between each support element and at least one longitudinal rail that extends from a first end of the stent to a second end of the stent. This type of secure, rigid spacing prevents the support elements from moving longitudinally along the rail(s) of the stent and prevents the stent from conforming to the curvature of the blood vessel in which it is deployed.

It is known to use a stent for controlled, time release therapeutic agent delivery within a vessel. For example, this concept is discussed in U.S. Pat. No. 5,102,417 to Palmaz and U.S. Pat. No. 5,464,650 to Berg, et al., both of which are hereby incorporated by reference in their entirety. These patents disclose different methods for applying agents, including therapeutic drugs, to a stent in order to reduce the incidence of restenosis, increase vascular healing and/or treat various conditions within the body in which the stent is deployed. However, the coatings of these stents are typically applied to an unexpanded stent. As a result, when the stent is expanded within the vessel, the coating is interrupted and separated, thereby causing portions of the stent to be left uncoated. This can result in an unreliable application of the agent within the vessel. Additionally, these stents and the coatings used to carry these agents can be very expensive to manufacture.

### SUMMARY OF THE INVENTION

The present invention is directed to an intraluminal stent having increased longitudinal flexibility when compared to prior art stents. Longitudinal flexibility as used herein relates to the flexibility of the stent structure (or portions thereof) to move relative to its major, longitudinal axis of extension.

An example of a stent according to the present invention includes a helically wound stent element freely mounted on a plurality of flexible rail elements that extend along the length of the stent. Because the stent element is freely mounted on the rail elements, various portions of the stent element are free to slide along the rail elements. Therefore, when the stent is placed in situ in a curved or otherwise bent blood vessel, the increased longitudinal flexibility of portions of the stent element along the axial extent of the stent allows those stent portions on the inside of the curve to move freely towards each other. On the other hand, the corresponding stent portions on the outside of the curve are free to move apart from each other. In this manner, the stent can more easily conform to the bend in the blood vessel and reduce the tendency of the stent to straighten the blood vessel.

In another embodiment of the present invention, the stent includes a plurality of independent radial vascular support elements freely mounted on at least one rail element. The behavior of the vascular support elements is similar to that of the helically wound stent structure as discussed above when the stent is bent or curved.

In yet another embodiment, the stent includes a plurality of vascular support elements each connected to at least one adjacent vascular support element and freely mounted on rail elements. This provides a consistent structural arrangement of the vascular support elements along the extent of the stent while still providing increased longitudinal flexibility in accordance with the present invention.

In still another embodiment, a stent according to the present invention comprises first and second terminal ends and a length extending between these terminal ends. The stent also includes at least one support rail extending between the terminal ends and a plurality of circumferentially extending support elements. The support elements each have an opening that receives the at least one support rail so that the support elements are moveable relative to the rail between the terminal ends of the stent.

The number of openings within each support element can correspond with the number of rails extended through the stent. The number and position of the rails is chosen so that the friction characteristic between the support elements and the interior of the blood vessel will be negligible when the relative direction of motion is in line with the bends, but increases when against the edge of the bent sections.

The present invention can include a lengthwise hole design with a solid wire rail, a flexible coil rail with a bent node stent or a combination of the two. The present invention also incorporates the use of a flexible coil as the rail, allowing rail expansion or contraction and preventing the ends of the rail from protruding past the ends of the stent, especially along the minor (inside) radius of curvature of a vessel. The present stent has a smooth profile. It also allows for reduced wall thickness and a smaller profile when compared to other embodiments of this invention, thereby allowing for less traumatic navigation through arteries, such as those of a human. The closed loop rail stents of the present invention retain the rails and provide even spacious distribution of the inter-ring linkages.

Another aspect of the present invention includes a stent for introducing within a body. The stent comprises a plurality of vessel support elements. A first one of the vessel support elements forms a first end support element and second one of the support elements forms a second end support element. The stent also comprises at least one support rail element extending between the end support elements. The support rail element includes a curved end section for extending beyond one of the end support elements. A plurality of the vessel support elements are moveable along and relative to the at least one support rail element.

### BRIEF DESCRIPTION OF THE DRAWINGS

The present invention will be even better understood with reference to the attached drawings, in which:

FIG. 1 illustrates a plurality of support elements according to the present invention;

FIG. 2 illustrates structural parameters of a respective support element according to the present invention that can be adjusted to provide different operational behaviors;

FIG. 3 illustrates the support elements of FIG. 1 mounted on rail elements according to the present invention;

FIGS. 4a-4c illustrate different geometries of the support elements according to the present invention;

FIGS. 5a and 5b illustrate hybrid combinations of geometries of the support elements according to the present invention;

FIG. 6 illustrates a variant geometry for the support elements according to the present invention, compared to that illustrated in FIG. 4a;

FIG. 7 illustrates an embodiment of the present invention including support elements, adjacent ones of which are joined together by at least one bridge element;

FIG. 8 illustrates a helically wound stent element mounted on rail elements according to the present invention;

FIG. 9 is an elevational view of a stent including a helically wound stent element;

FIG. 10 is a perspective view of a stent according to the present invention including a plurality of support elements;

FIGS. 11a-11c illustrate various examples of rail end structures for preventing support elements from becoming disengaged or dismounted from rail elements according to the present invention;

FIG. 12 is a perspective view of the stent according to another embodiment of the present invention;

FIG. 13 is a side view of the stent of FIG. 12;

FIG. 14 is a perspective view of the stent illustrated in FIG. 12;

FIG. 15 is an end view of the stent of FIG. 12;

FIG. 16 is a side view of the stent shown in FIG. 12;

FIG. 17 is a cross section of a rail according to the present invention;

FIG. 18 is an elevational view of an alternative embodiment of a stent according to the present invention having rails with curved end sections;

FIG. 19 is an elevational view of the stent shown in FIG. 18 with multiple, independent rail elements having terminal ends fixed to outer vessel support members;

FIG. 20 illustrates the stent shown in FIG. 18 in an unexpanded state;

FIG. 21 illustrates the stent shown in FIG. 18 in an expanded state; and

FIGS. 22-24 illustrate alternative embodiments of the stent shown in FIG. 18 with different rail arrangements.

### DETAILED DESCRIPTION OF THE INVENTION

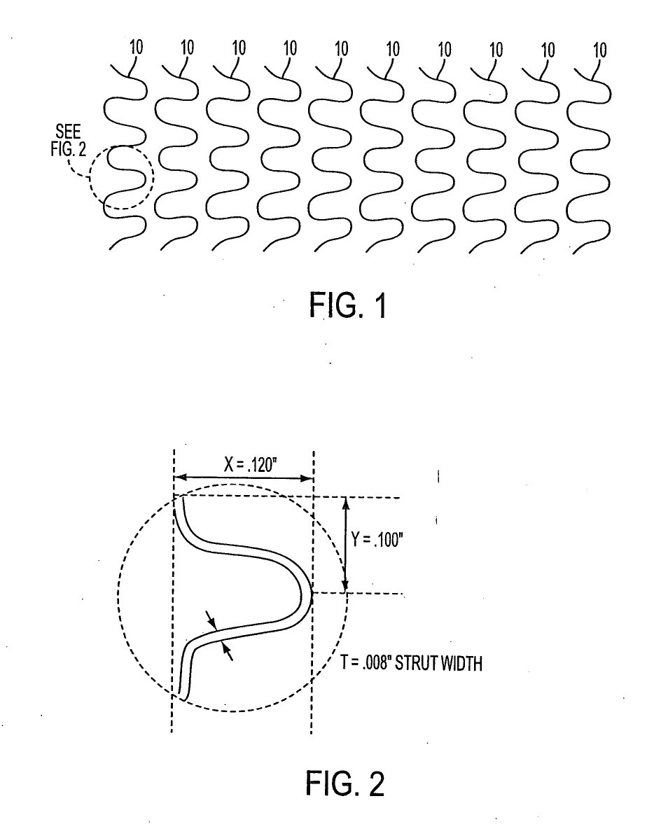

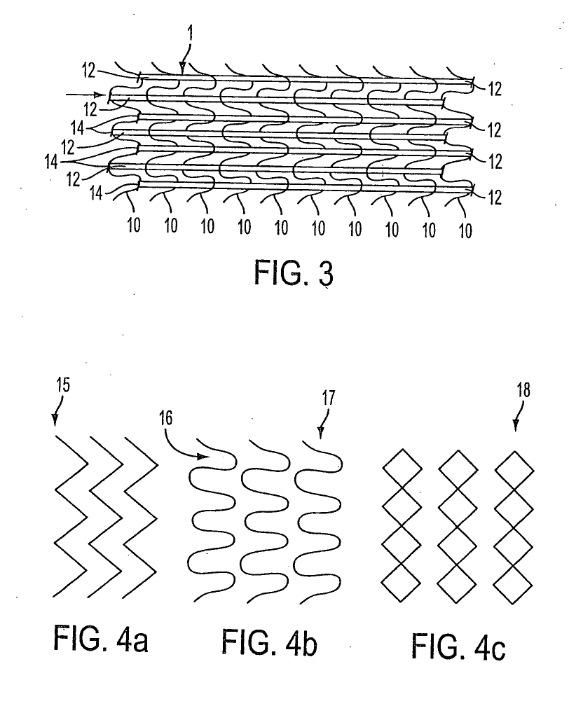

Referring to the figures where like numerals indicate the same element throughout the views, FIG. 1 illustrates a representative structure of vascular support elements 10 (also referred to as “hoop elements”) for forming a portion of a stent 1 (see, for example, FIG. 3) according to the present invention. As discussed below, the support elements 10 act as radial supports for the vessel in which they are deployed. Each support element 10 is generally annular in shape. However, for the purpose of illustration, each support element 10 is depicted two-dimensionally on the paper as though it has been cut and laid flat.

Each support element 10 is made from a flexible, biocompatible material (i.e., from a material that is, for example, non-reactive and/or non-irritating) including those discussed below with respect to the rails of the present invention. In one example of the present invention, support elements 10 are made from medical-grade metal wire formed as a closed loop (i.e., as an annular hoop) in a known manner, including, for example, micro-welding two ends of a wire segment together. Stainless steel, metal alloys, and polymeric materials used in conventional stents are representative examples of materials from which support elements 10 can be formed. The polymers for support elements 10 may, for example, be bioabsorbable polymers so that the stent can be absorbed into the body instead of being removed. As discussed below, these materials also include super elastic alloys such as Nitinol.

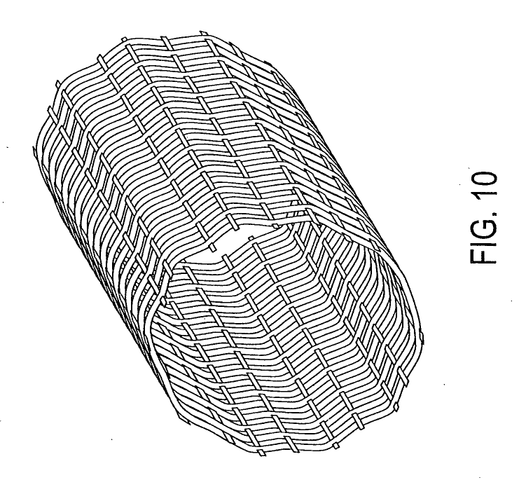

Preferably, each support element 10 has a sinusoidal or otherwise undulating form, such as the rounded wave shape seen in FIG. 1 by way of example. As shown in FIG. 1, the undulating form of the support elements includes peaks 11 and troughs 13 (space behind the peaks). Each peak points in a direction that is opposite that of the immediately, circumferentially positioned peak 11. The same is true of the valleys 13. The direction of undulation may be axial, as illustrated in FIG. 1, or radial, as seen, for example, in FIG. 10.

As seen in FIG. 2, certain parameters of support elements 10 can be altered in order to adjust the operational behavior of the stent 1. For example, as seen by way of example in FIG. 2, the wave height X and the peak-to-trough distance Y can be made larger or smaller, and/or the thickness T of the support element 10 can be made thicker or thinner. For example, X may be about 0.120 inch, Y may be about 0.100 inch, and T may be about 0.008 inch. However, other dimensions can also be used depending on the needs of the particular stent.

FIG. 3 illustrates support elements 10 freely mounted on rail elements 12. Rail elements 12 are desirably sufficiently flexible to accommodate bends, curves, etc. in a blood vessel. In contrast to bridge elements 28 which are generally the same thickness and the hoops 10 that they join and thus relatively inflexible, the thickness of the rail elements 12 can be designed to provide a desired degree of flexibility to a given stent.

Rail elements 12 may be made from, for example and without limitation, metals, metallic alloys, glass or acrylic, and polymers. Additional materials from which the rails 12 of the present invention or the support elements 10 can be formed are discussed below.

As seen in FIG. 3, each rail element 12 is “woven” between adjacent support elements 10. In particular, each rail element 12 passes alternately inside and outside (or over and under, as seen in this depiction) adjacent support elements 10. Each rail element may, for example, be passed inside/outside (or under/over) adjacent support elements 10 at the respective peaks thereof, as illustrated in FIG. 3. Thus, adjacent longitudinally extending rail elements alternate inside and outside of a given support element 10 along a circumferential direction thereof. This increases the structural integrity of the stent and helps resist lateral crushing forces that may be applied to the stent. As with the other embodiments discussed herein, the support elements can have any known shape and pattern.

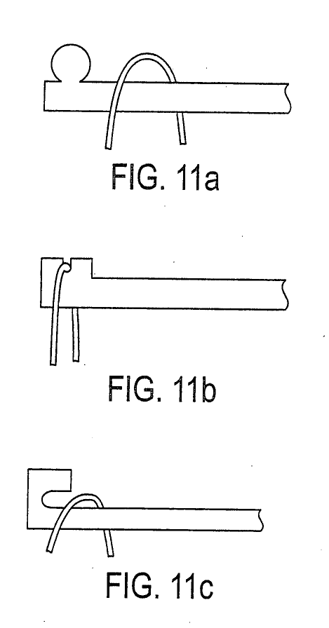

At least some of rail elements 12 may include end structures 14 for preventing the support elements 10 from unintentionally passing beyond the ends of the stent 1. End structures 14 may have several forms as illustrated in FIGS. 11a- 11c. For example, end structures 14 may be mechanical stop members mounted on the ends of each rail element 12 to block the freely mounted support elements 10 from being dismounted from rail elements 12, effectively keeping the support elements 10 from “falling off” of the ends of rail elements 12. Examples of mechanical stop elements include balls or other protrusions formed at the ends of each rail element 12 that act as stops (see, for example, FIG. 11a).

Alternative mechanical stop elements usable with the disclosed embodiments include slotted members at the end of each rail as shown in FIG. 11c. Additionally, the stop elements could include mechanical deformations on the ends of the rail elements 12 or members, such as caps, secured to the terminus of the rail element 12 that are larger than the openings in the support elements that receive the rail elements 12. The discussed stop elements can be used with any of the embodiments discussed herein. In each of the above-discussed embodiments, all of the support elements 10 are free to move along the length of the rail elements 12, to the extent permitted by the mechanical stop elements.

In another example, end structures 14 may be a mechanical grasp structure by which the endmost support elements 10 are fixed in place relative to the ends of rail elements 12 (although the remaining support elements 10 remain freely mounted on rail elements 12). See, for example, FIG. 11b.

End structures 14 may also be (depending on their intended effect), a suture or other ligature by which a portion of an endmost support element 10 is tied to the end of rail element 12, or a weld (made by, for example, a laser) for bonding a portion of an endmost support element 10 to an end of rail element 12.

FIGS. 4a-4c, 5a-5b, 6, and 8 illustrate examples of geometries for the support elements 10. The geometries illustrated have different advantages.

For example, the geometry 15 illustrated in FIG. 4a is useful for forming support elements in self-expanding stents formed of Nitinol, because it allows better crimping in preparation for insertion.

The geometry in FIG. 4b, which its relative wide “trough” portions 16 may, for example, facilitate engagement with a respective rail element 12 in the manner discussed above.

The diamond-shaped geometry 18 in FIG. 4c could be considered a variant of the sawtooth geometry shown in FIG. 4a. Like the example shown in FIG. 4a, the diamond-pattern in FIG. 4c is useful for self-expanding stents formed of Nitinol because it facilitates crimping. In addition, it offers increased torsional rigidity and greater surface structure for support.

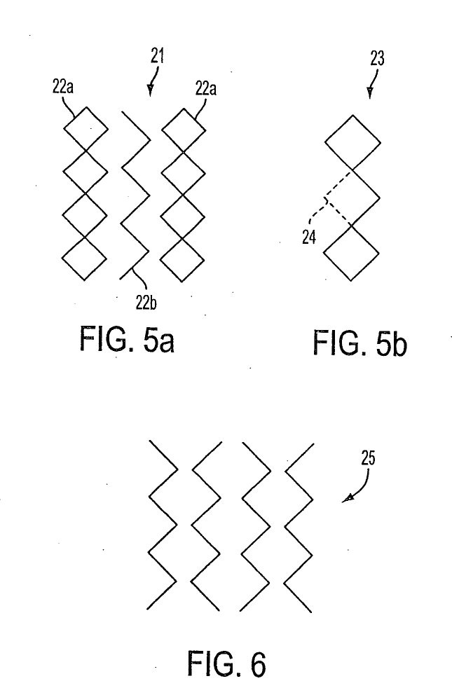

The geometries 21 and 23 of FIGS. 5a-5b, respectively, may, for example, be useful in covered stents or in stent-grafts requiring less scaffolding. Here, “scaffolding” refers to the amount of supporting structure in a given portion of the stent. For example, the combination of two diamond-shaped support elements 22a plus a sawtooth support element 22b does not provide as much supporting structure as three diamond-shaped support elements. Likewise, a diamond-shaped geometry 22 having part of some of the diamonds omitted (as indicated in phantom at 24) provides less supporting structure than support elements including complete diamonds.

The geometry 25 illustrated in FIG. 6 appears to have comparatively increased longitudinal flexibility and may permit specialized interaction with rail elements 12 in terms of force distribution and the like.

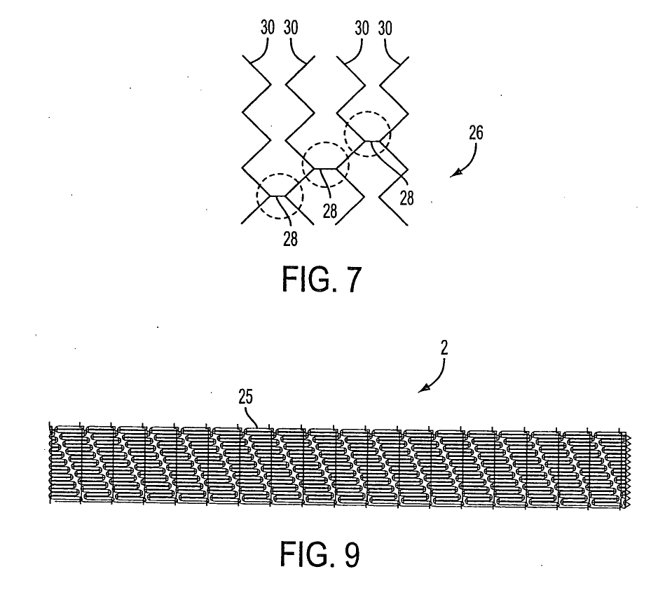

The geometry 26 illustrated in FIG. 7 includes at least one bridge element 28 between adjacent support elements 30 that like support elements 10 act as radial support elements for the vessel in which they are deployed. However, unlike the hoop elements 10 of the embodiment illustrated in FIG. 3, the hoop elements are not independent from one another, as discussed above. Providing at least one bridge element 28 between adjacent support elements increases the structural integrity of the stent because it helps to keep the support elements 30 distributed along the length of the stent while still offering increased longitudinal flexibility.

Preferably, only a limited number of bridge elements 28 are provided between respective adjacent support elements. If too many bridge elements 28 are provided between adjacent support elements, the coupling there between becomes similar to providing a rigid coupling there between, such that the desired longitudinal flexibility according to the present invention is lost. By providing only a limited number of bridge elements 28 (including, without limitation, one bridge element 28), the resultant assembly can still provide a good approximation of using completely independent support elements.

Furthermore, the peripheral location at which bridge element(s) 28 are provided between respective adjacent support elements has an effect on longitudinal flexibility. For example, if two bridge elements are provided between a respective pair of adjacent support elements at diametrically opposite sides of the support elements, then, generally, the longitudinal flexibility there between is at a maximum at diametrically opposite sides of the support elements located at about 90 degrees from the bridge elements, and decreases along the circumference of the support elements in a direction approaching the respective bridge elements.

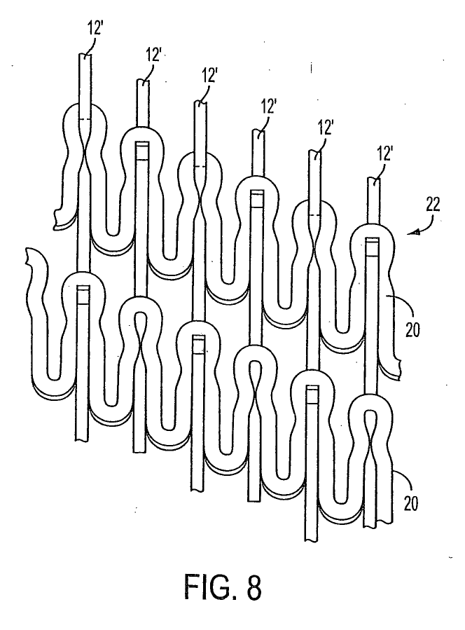

For the foregoing reasons, it may be useful or otherwise beneficial to provide, for example, one bridge element 28 between adjacent support elements 30, as illustrated in FIG. 8. Furthermore, it may be additionally useful to offset each bridge element 28 from an adjacent bridge element 28 along a circumferential direction, as is also illustrated in FIG. 7. This circumferential offset provides the structural integrity benefits of using a bridge element 28, but distributes the resultant restriction in longitudinal flexibility so that no one transverse direction of stent deflection is overly restricted.

As mentioned above, instead of using independent support elements 10 to provide radial support to a vessel, a single helically wound stent element 20 can be freely mounted on one or more substantially parallel rail elements 12′ as seen in FIG. 8 to act as a radial support element for the vessel in which it is deployed. As with the support elements 10, rail elements 12′ are woven over/under respective portions of the stent element 20, such as, for example, over/under the respective peak portions.

FIG. 8 also illustrates a feature of the invention that is applicable to both the support elements 10 and the helically wound stent element 20. Specifically, a portion of, for example, stent element 20 adjacent to a respective peak portion is pinched in, or necked in, and a respective rail element 12′ is passed through the restricted portion 22 defined thereby. This advantageously limits relative movement between stent element 20 and rail element 12′. This maintains the relative alignment of rail elements 12′ and, as a result, increases the structural integrity and the overall hoop strength of the stent. It will be appreciated that instead of a pinched or a necked portion 22, an end portion of each peak portion could simply have a suitably sized hole (not shown here) formed there through which would be similar to closing the pinched in peaks shown in FIG. 8.

As mentioned above, the concept of a restricted portion 22, as seen in FIG. 8, is equally applicable to the arrangement of, for example, FIG. 3.

FIG. 9 is view of an entire stent 2 using a single helically wound stent element 25. It can be appreciated from FIG. 9 that a helically wound stent element, such as that illustrated at 25, has some effective similarity to a plurality of obliquely extending independent support elements. However, instead of using bridge elements (in the manner discussed with respect to FIG. 7), the use of a single stent element addresses at least some of the issues raised above with respect to longitudinal structural integrity.

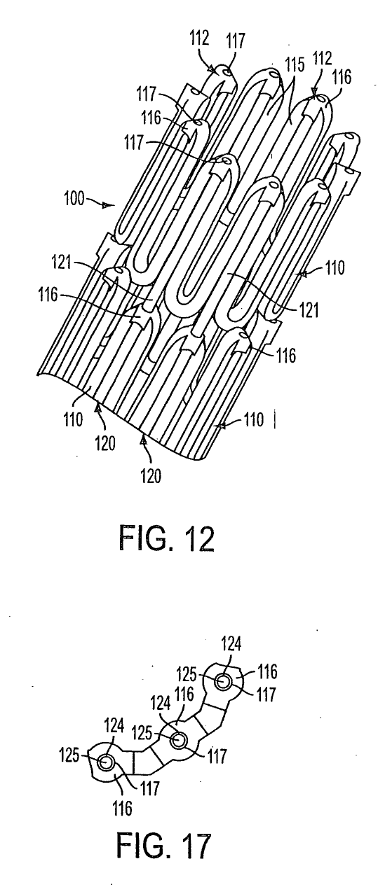

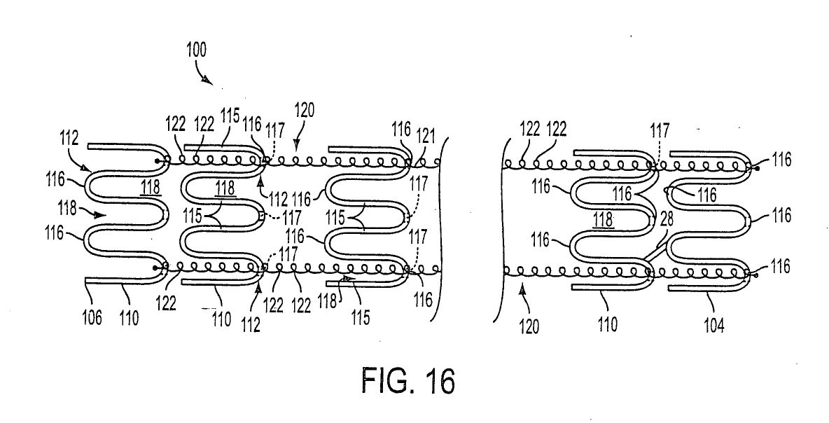

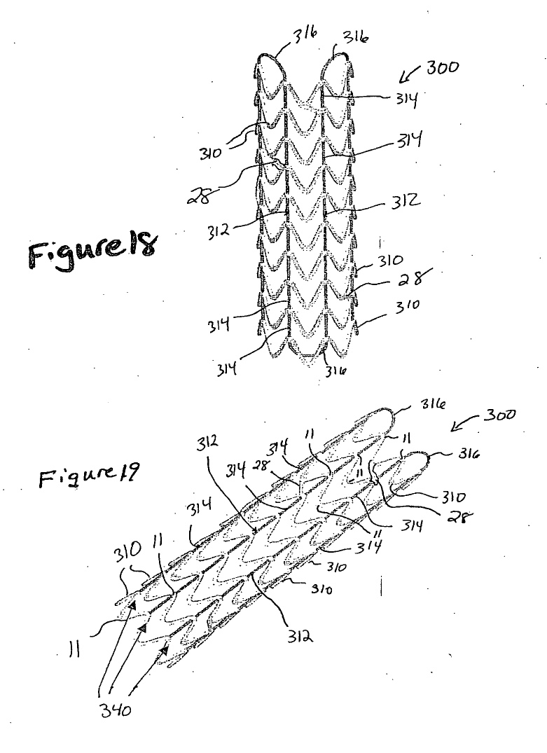

An additional embodiment of the stent 100 according to the present invention is illustrated in FIGS. 12-16. Like the embodiments discussed above and illustrated in FIGS. 3 and 8, the stent 100 illustrated in FIGS. 12-16 includes a plurality of support elements 110 spaced a long its length. These support elements 110, like those discussed above, provide radial support to a blood vessel after the stent 100 has been deployed into a mammalian body and expanded. As with the other stents discussed above, stent 100 can be expanded by conventional techniques such as an inflatable balloon positioned within the stent 100.

As seen in FIGS. 12-15, the support elements 110 have the same general shape as those discussed above. Support elements 110 are generally annular in shape and have a generally hoop-like appearance. Hence, support elements 110 will be referred to as support elements 110 below. Adjacent support elements 110 are spaced from each other by bridge element 28 in the same manner as discussed above. Also, each support element 110 is formed of a flexible, biocompatible material such as those discussed above. As with the other stents, the stent 100 can be formed of a metal, metal alloy such as Nitinol, or polymer, etc.

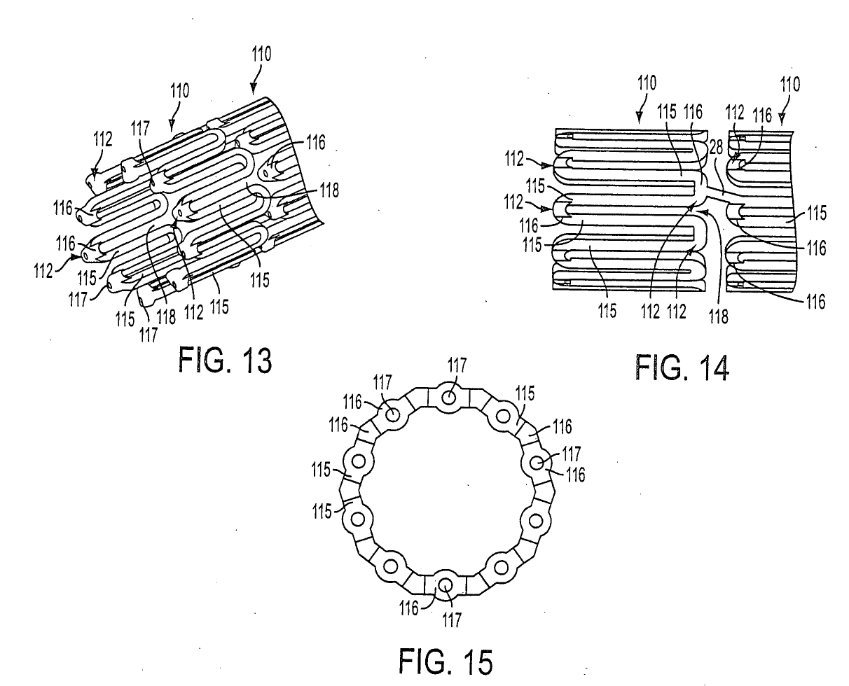

As seen in FIGS. 12-14, the support elements 110 have a generally sinusoidal or otherwise undulating form. As shown in FIGS. 13 and 14, the undulating form of the support elements 110 is comprised of a plurality of substantially longitudinal struts 115 and a plurality of curved connecting members 116. Each curved member 116 connects adjacent longitudinal struts 115 together to form the continuous support element 110. Each curved connecting member 116 forms a peak 112 along the alternating path of each hoop 110. A trough 118 is formed at the end of each longitudinal strut 115 opposite the peak 112. Troughs 118 include the open spaces between adjacent longitudinal struts 115 that are connected to the same curved member 116 at a respective peak 112. As seen in FIG. 12, each peak 112 points in a direction that is opposite to that of the immediately proceeding peak 112 along the circumference of each hoop. Conversely, each peak 112 points in the same direction as the adjacent longitudinally spaced peak 112. The same is true of the troughs 118. For example, the troughs 118 are open in a direction opposite that of the immediately adjacent troughs 118 around the circumference of the hoop 110.

As with the other embodiments discussed above, stent 100 also includes at least one rail element 120 (hereinafter “rail”) that extends from a first terminal end 104 to a second terminal end 106. Each end 104, 106 is formed by one of the support elements 110 secured to the rail(s) 120. As illustrated in FIG. 12, the stent 100 can include two rails 120 that extend between the ends 104, 106. It is also contemplated that any number of rails 120 up to the number of peaks 112 along a support element 110 could be used. For example, if the support elements 110 include ten peaks 112, then up to ten rails 120 could be used. In between the hoops 110 at the terminal ends 104, 106, the remaining hoops 110 that are connected to each other by the bridge elements 28 are free to move along the rail(s) 120. These remaining hoops 110 slide along the rail(s) 120 so that the stent 100 can conform to the shape of the blood vessel.

Unlike the support elements 10, the support elements 110 include apertures 117 in the curved members 116 through which the rails 120 extend as shown in FIG. 12. Apertures 117 extend through the peaks 112 in a direction that is substantially parallel to the length of the stent 100. These apertures 117 retain and orient the supporting rail(s) 120 in a direction parallel to the length of the stent 100. Also, the rails 120 are completely contained within the walls (within the outer surface) of the stent 100. These walls form the apertures 117. By positioning the rails 120 extending within the walls of the stent 100, the rails 120 are not alternately woven from an inside surface to an outside surface of the stent 100 or in another way that could compromise the straightness of the rail 120.

In an embodiment of stent 100, the rail 120 is made of a flexible coil spring 121 instead of a solid wire. The flexible coil spring 121 is coiled a bout an axis that extends parallel to the longitudinal axis of the stent 100 before it is deployed within a blood vessel. When the stent 100 is straight, the coil spring 121 is at rest. As a result, the coil spring 121 is not under tension and no longitudinal pressure is applied to the hoops 110 by the coil spring 121. However, the coils 122 of the coil spring rails 121 are spaced from each other along the length of the stent 100 so that the coils 122 of the spring 121 can collapse upon themselves and shorten when and where needed. For example, when the stent 100 is deployed into a curved vessel, the stent 100 will conform to the curve of the blood vessel without straightening the vessel. This is accomplished by the coils 122 along the minor curve of the vessel compressing to a shorter length than when the stent 100 is at its rest length. The coil spring 121 aids in providing the shortest possible stent length on the minor radius of curvature of the vessel. Along the major curve, the coil spring 121 remains at rest or expands, and allows the stent 100 to follow the curve of the vessel.

In an alternative embodiment, the coil spring 121 is slightly extended and under tension when straight before deployment. As a result, the stent 100 is under a slight compressive force prior to deployment. This slightly compressive force assists in the stent 100 conforming to the minor curve of the vessel. In either of the above embodiments, the hoops 110 are spaced and held relative to each other by the bridge elements 28. In the second embodiment, the bridge elements 28 prevent the collapse of the stent 100 under the pressure of the coil spring 121.

In another embodiment, a solid wire rail 125 is used in conjunction with the flexible coil spring 121. As illustrated in FIG. 17, the solid rail 125 runs down the lumen 124 of the coil spring 121 providing structural support to the coil spring 121. Multiple rails 125 can also be positioned within the lumen 124 of a coil spring 121. In still another embodiment, the rail 120 is a flexible or substantially rigid elongated rod.

The struts 115 of stent 100 can have substantially any radial thickness that provides them with the needed strength to support a blood vessel while still achieving a low profile that will not damage the vessel as it is deployed. In one example, the struts 115 can have a radial thickness of between about 0.002 inch and about 0.008 inch. In another preferred embodiment, the struts 115 have a radial thickness of between about 0.004 inch and about 0.005 inch. These thicknesses provide the stent 100 with the needed structural and expansion properties to support a vessel and conform to its shape. Additionally, the areas of the curved members 116 must be formed with a greater radial thickness than the struts 115 in order to accommodate the apertures 117. For example, the radial thickness of the curved members 116 can be between about 0.001 inch and about 0.006 inch greater than that of the struts 115. The apertures 117 can have a diameter of about 0.005 inch for receiving the rails 120. Between the rails 120 where expansion occurs, the thickness could be about 0.004 inch. A stent 100 having 0.002 inch thick strut 115 walls could have a curved member 116 with a radial thickness of about 0.009 inch where the rails 120 are woven.

In one embodiment, the process for manufacturing the stent 100 includes the steps of providing a hypotube that has a number of small lumens through its wall that form apertures 117 and machining the tube to a form a ring precursor. The lumens and the form of each support element 110 are then laser cut for speed and accuracy. For example, a laser can cut the stent pattern and align the apertures 117 at the peak 112 of the sine wave. Also, the laser provides the support elements 110 with a smooth profile. As is understood in the art, the support elements 110 should be void of jagged edges because they will damage the vessel and/or not deploy properly. Other known ways of forming these support element hoops can also be used with the present invention. For example, the stent 100 could be produced using metal extrusion, hot or cold pulling over a fixture of wires, metal injection molding and welding tube assemblies. The supporting rails 120 maintain a relatively smooth profile that has a consistently low friction characteristic in both directions of motion.

Alternative embodiments of a stent that facilitates insertion into a vessel according to the present invention are illustrated in FIGS. 18-23. These alternative embodiments include different rail element assemblies that provide additional rail element geometries with respect to the embodiments discussed above. Like the embodiments discussed above, the embodiments illustrated in FIGS. 18-23 each include a stent 300 having a plurality of spaced vascular support elements 310 that include any one of the support elements illustrated in FIGS. 1-17.

Adjacent vascular support elements 310 can be free of attachment to each other or connected by bridge elements 28 as discussed above. The stent 300 also includes at least one elongated rail element 312 that has at least one elongated section 314 extending parallel to the length of the stent 300. As shown in FIG. 18, a preferred embodiment of the rail element 312 is a continuous member that includes a plurality of curved rail loop end sections and a plurality of elongated sections 314 that extend parallel to each other and the length of the stent when the stent is in at least its undeployed state (see FIG. 20).

As shown in FIG. 18, adjacent elongated sections 314 are connected together by respective curved end sections 316 so that the rail element 312 forms the continuous, uninterrupted piece of elongated sections 314 and curved loop sections 316 that extend along the different axes of the stent 300. These curved end sections 316 do not cause friction or trauma to the vessel during introduction, tracking or deployment of the stent 300. The curved end sections 316 can be provided at both ends of the stent 300 as shown in FIGS. 18 and 21. Additionally, as discussed below and shown in FIG. 19, the curved end sections 316 permit the terminus 340 of each elongated section 314 that is not integral with a curved end section 316 to be fixed to the outer support element 310 at the inner peaks 11 that are spaced from the outer peaks 11 of the outermost vessel support element 310. As a result, the termini 340 of the elongated sections 314 do not extend beyond the outermost longitudinal point of the outer vessel support element 310 (as shown in FIG. 19). This provides the stent 300 with a leading edge that is free of rail protrusions and that facilitates deployment of the stent 300 into a vessel. As with the other embodiments discussed herein, the support elements 310 can be secured to the rail elements 312 by welding, including spot welding, and other known fixation techniques. The rail elements 312 can further include swaging end(s), looping end(s), tying end(s) and welding of the elongated section 314 back onto itself or another elongated section 314.

In an alternative embodiment shown in FIG. 19, the stent 300 can include two or more rail elements 312. In this embodiment, each rail element has two or more elongated sections 314 that are connected to each other by one or more curved end sections 316. However, unlike the embodiment disclosed in FIG. 18, the rail elements 312 are only directly connected to the support elements 310 at their termini 340 and not to each other. In a preferred embodiment the rail ends are secured to each other such that all support elements 310 of the stent are free floating.

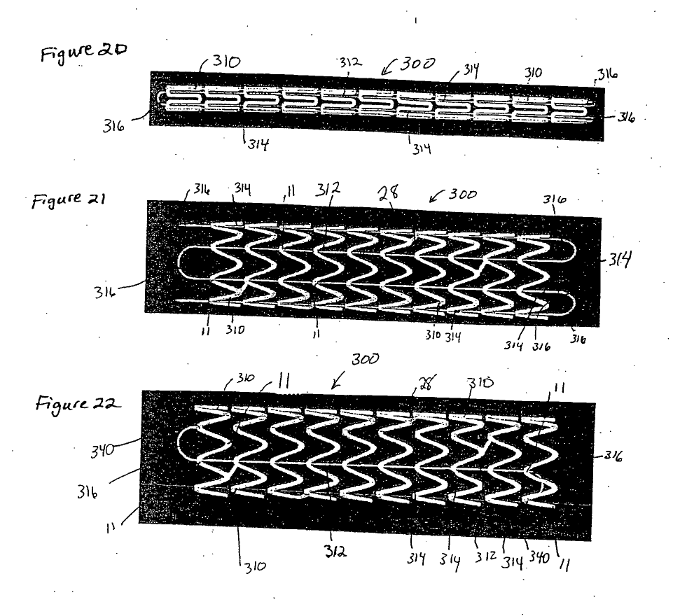

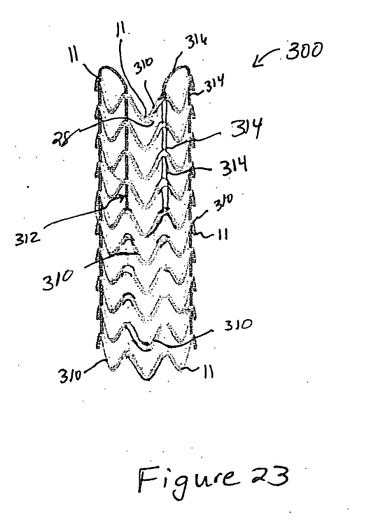

The embodiment of the stent 300 illustrated in FIG. 22 includes at least one rail element 312 that each have a single elongated section 314 and a curved end section 316 extending from each elongated section 314. In this embodiment, each outer support element 310 is secured to the rail element(s) 312 at the transition area between the elongated section 314 and the curved end section 316 and/or at the terminus of the curved end section 316. In this embodiment, the elongated sections 314 could alternatively be integral with a curved end section 316 at one end and directly connected to an outer support element 310 at the other.

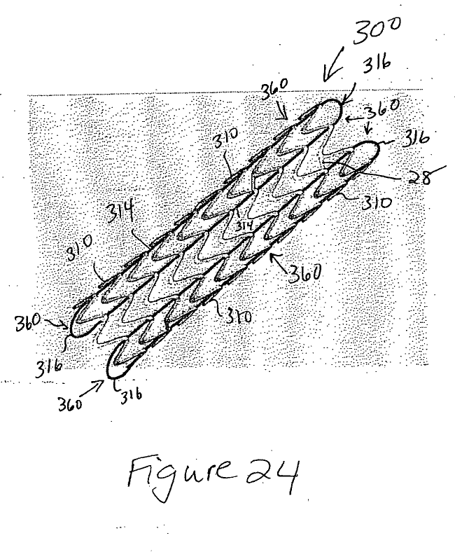

In yet another embodiment illustrated in FIG. 24, the stent 300 has at lease one rail element 312 that forms a closed loop 360. The closed loop is formed of two elongated sections 314 that are connected to two opposing end sections 316. Each stent 300 may incorporate multiple closed loops 360 as desired. Additionally, as with the other embodiments discussed herein, the loop 360 may be completely free floating on all support elements 310 or may be optionally secured to one or more support elements as desired. As a result, the support elements 310 of each embodiment are unsecured to the elongated sections 314 and thus can move anywhere along the elongated members 314 and the loop end sections 316.

In any of the above-discussed embodiments, the curved end sections 316 form continuous, semi-circular ends at one or both ends of the stent 300 that operate to prevent the support elements 310 from separating from the stent 300 or its rail elements 312. As a result, the support elements 310 do not need to be secured to the rail elements 312 along the elongated sections 314 and between the curved end sections 316. This permits the support elements 310 to move freely along the length of the stent 300 and between the termini of the elongated sections 314 and/or the curved end sections 316. The support elements 310 are free of any secure connection to the rail elements 312 that extend through them so that the stent 300 can conform to the shape of the vessel in which it is deployed.

In any of the discussed embodiments, the terminus of a rail element 312, be it the end of an elongated section 314 or the end of a curved section 316, does not need to be located at, and secured to, the outermost, terminal support elements 310. Instead, the terminus of one or more rail elements 312 can be connected to a support element 310 positioned between the outer, terminal support elements 310 as shown in FIG. 23. The positioning of this terminus of one or more rail elements 312 also provides the ability to achieve a predetermined curve to the stent 300 upon its expansion. In this embodiment, the predetermined foreshortening of the stent can be predetermined and effected by securing the support elements 310 to the rail elements 312 including the terminus of the rail element 312 at predetermined locations that will result in one side of the stent 300 assuming a predetermined smaller curve than the other side of the stent 300 upon expansion.

The elongated rail element 312 comprised of the straight sections 314 and the curved sections 316 reduces the production and assembly process of the stent 300. Additionally, these rail elements 312 permit the body of the stent 300 to have a predetermined bend that matches the curve of the vessel into which it is deployed. In any of the above-discussed embodiments, the curved rail sections 316 can be solid or formed of springs or coils. The rail elements 312 can be formed of any of the materials discussed herein. Moreover, in any of these embodiments that include curved end sections 316, the curve in the rail elements 312 provide low friction rail ring support that allows the stent 300 to move easily within a lumen before its expansion and provides non traumatic tissue interaction after its expansion.

The rail elements 12, 12′, 120 and 320 according to the present invention may be fabricated from a variety of biocompatible materials, including metals, alloys, and metallic compounds (e.g., metal oxides), polymers (e.g., resins), amorphous materials (e.g., ceramics, silica, and glassine), carbons (e.g., pyrolytic carbon, such as the coating Carbofilm™, amorphous carbon, activated carbon, and fullerenes as described, e.g., in WO 01/68158) and others. In general, suitable materials will exhibit biocompatibility, sufficient flexibility to navigate lumens during insertion, and the ability to contact and be secured relative to the vascular lumen wall. The term “biocompatible” refers to materials that do not have toxic or injurious effects on biological systems. Thus, the stents should not substantially induce inflammatory and neointimal responses. Any of the biocompatible materials discussed below may be used as the primary material to form the rails or other portions of the disclosed stents, or may be used to form a film, coating, or layer to cover a base material (e.g., a metal) that may or may not be biocompatible. Coating techniques are known in the art and are described, e.g., in U.S. Pat. No. 6,153,252. If the stent material covers a base material that is itself biocompatible, complete coating of all exposed surfaces of the base material may not be necessary.

Preferably, the stent rails 12, 12′, 120 and 312, along with other portions of the discussed stents, comprise biocompatible metals, metal alloys, and biocompatible polymers. For example, a type of biocompatible polymer usable with the stents according to the present invention includes the resilient polymeric materials disclosed in international publication WO 91/12779. Additional biocompatible metals and alloys include those disclosed, e.g., in U.S. Pat. Nos. 4,733,665; 4,800,882; 4,886,062; and 6,478,815. Such metals and alloys include, but are not limited to, silver, tantalum, stainless steel, annealed steel, gold, copper alloys, cobalt alloys (e.g., cobalt-chromium-nickel alloys), titanium, tungsten, zirconium, niobium, iridium, and platinum. Shaped-memory metal alloys (e.g., Nitinol, a super elastic titanium alloy) can also be used to form the rails discussed herein.

Biocompatible polymers that are used with the rails 12, 12′, 120 and 312 of the present invention can be nonbioabsorbable, bioabsorbable in part, or substantially completely bioabsorbable. The stable, nonbioabsorbable polymers that may be used for stent rail construction are those generally exhibiting a low chronic tissue response. These include polyesters, polyamides, polyolefins (substituted or unsubstituted with e.g., halides), polyurethanes (e.g., polyurethane urea, segmented polyurethane urea/heparin) and silicones (e.g., siliconeA, siliconeB, and silicone C) (see, e.g., van Beusekom et al. Circulation 86(suppl I):I-731, (1992) and Lincoffet et al. J Am. Coll Cardiol 21: 886, 887 (1993).

Polyesters include e.g., polyethylene terephthalate (PET) and polybutylene terephthalate (PBT). Other polyesters include polyethylene terephthalate copolymers or polybutylene terephthalate copolymers using, as comonomers, saturated dibasic acids such as phthalic acid, isophthalic acid, sebacic acid, adipic acid, azelaic acid, glutaric acid, succinic acid, and oxalic acid; polyethylene terephthalate copolymers or polybutylene terephthalate copolymers using, as diol comonomers, 1,4-cyclohexanedimethanol, diethylene glycol, and propylene glycol; and blends thereof. Specific examples of these polyethylene terephthalate copolymers include polyethylene terephthalate/isophthalate (PET/I), polyethylene terephthalate/sebacate (PET/S), and polyethylene terephthalate/adipate (PET/A). Specific examples of the polybutylene terephthalate polymers include polybutylene terephthalate (PBT), polybutylene terephthalate/isophthalate (PBT/I), polybutylene terephthalate/sebacate (PBT/S), polybutylene terephthalate/adipate (PBT/A), polybutylene/ethylene terephthalate, and polybutylene/ethylene terephthalate/isophthalate. Also usable are polyesters that are copolymerized or modified with other third components in order to improve their physical characteristics. The polyester resins may be stretched either monoaxially or biaxially.

Polyamides include, e.g., polyamides, Nylon 66, polycaprolactam, and molecules of the form —NH—(CH₂)ₙ—CO— and NH—(CH₂)ₓ—NH—CO—(CH₂)_{y}—CO, wherein n is preferably an integer in from about 6 to about 13, x is an integer from about 6 to about 12, and y is an integer from about 4 to about 16.

Polyolefins include, e.g., polypropylene, polyethylene, polyisobutylene, polytetrafluoroethylene, expanded polytetrafluoroethylene, ethylene-alphaolefin copolymers. Polyolefins also include copolymers of olefins and unsaturated glycidyl group-containing monomers, and terpolymers or multipolymers of olefins, unsaturated glycidyl group-containing monomers and ethylenically unsaturated monomers. Examples of olefins include propylene, butene-1, hexene-1, decene-1, octene-1. Examples of the unsaturated glycidyl group-containing monomers include e.g., glycidyl esters such as glycidyl acrylate, glycidyl methacrylate, monoglycidyl itaconate, monoglycidyl butenetricarboxylate, diglycidyl butenetricarboxylate, and triglycidyl butenetricarboxylate; glycidyl esters of α-chloroallyl, maleic acid, crotonic acid, and fumaric acid; glycidyl ethers such as vinyl glycidyl ether, allyl glycidyl ether, 2-methyallyl glycidyl ether, glycidyloxyethyl vinyl ether, and styrene-p-glycidyl ether; and p-glycidylstyrene. In addition to olefins, other ethylenically unsaturated monomers of the invention may also be used to form homo- or copolymers. Such monomers include, e.g., vinyl esters and α- and β-ethylenically unsaturated carboxylic acids and derivatives thereof. Examples include vinyl esters such as vinyl acetate; vinyl propionate; vinyl benzoate; acrylic acid; methacrylic acid and esters thereof, such as methyl, ethyl, propyl, butyl, 2-ethylhexyl, cyclohexyl, dodecyl, and octadecyl acrylates or methacrylates; maleic acid; maleic anhydride; itaconic acid; fumaric acid; maleic mono and diesters; vinyl chloride; vinyl ethers such as vinyl methyl ether and vinyl ethyl ether; and acrylic amides.

Other useful nonbioabsorbable polymers include poly(meth)acrylates, polyalkyl oxides (polyethylene oxide), polyvinyl alcohol homo- and copolymers (e.g., PVA foams, polyethylene vinyl alcohol), polyethylene glycol homo- and copolymers, polylysine, polyoxamers, polysiloxanes (e.g., polydimethylsiloxane), polyethyloxazoline, and polyvinyl pyrrolidone, as well as hydrogels such as those formed from crosslinked polyvinyl pyrrolidinone and polyesters (e.g., polyvinyl pyrrolidone/cellulose esters and polyvinyl pyrrolidone/poly urethane). Further nonbioabsorbable polymeric materials include acrylic polymers (e.g., methacrylate) and copolymers, vinyl halide polymers and copolymers (e.g., polyvinyl chloride), polyvinyl ethers (e.g., polyvinyl methyl ether), polyvinylidene halides (e.g., polyvinylidene fluoride and polyvinylidene chloride), polymethylidene maleate, polyacrylonitrile, polyvinyl ketones, polyvinyl aromatics (e.g., polystyrene), polyvinyl esters (e.g., polyvinyl acetate), copolymers of vinyl monomers with each other and olefins (e.g., etheylene-methyl methacrylate copolymers, acrylonitrile-styrene copolymers, ABS resins and ethylene-vinyl acetate copolymers), alkyd resins, polycarbonates, polyoxymethylenes, polyimides, polyethers, epoxy resins, rayon, rayon-triacetate, cellulose, cellulose acetate, cellulose acetate butyrate, cellophane, cellulose nitrate, cellulose propionate, cellulose ethers (e.g., carboxymethyl cellulose and hydoxyalkyl celluloses), cellulose esters, and combinations thereof.

Bioabsorbable polymers may also be used for the stent rails 12, 12′, 120 and 312 and other parts of the stents of the present invention. Bioabsorbable polymers are advantageous in that stents or portions thereof formed from these materials can be absorbed into the body and therefore do not require physical removal. Bioabsorbable polymers include, for example, those found in Tanquay et al. (Contemp. Intervention. Tech. 12(4):699-713, (1994)). Bioabsorbable polymers differ from nonbioabsorbable polymers in that they can be degraded into substantially non-toxic biodegradation products, while used in in vivo therapy. Degradation generally involves breaking down the polymer into its monomeric subunits. For example, the ultimate hydrolytic breakdown products of a poly(phosphonate) are phosphonate, alcohol, and diol, all of which are potentially non-toxic. The rate of degradation of bioabsorbable polymers is related to various polymer properties, such as permeability, water solubility, crystallinity, and physical dimensions.

Bioabsorbable polymers include various types of aliphatic polyesters, polyorthoesters, polyphosphazenes, poly(amino acids), copoly(ether-esters), polyalkylene oxalates, polyamides, poly(iminocarbonates), polyoxaesters, polyamidoesters, polyoxaesters containing amido groups, poly(anhydrides), poly(hydroxybutyrates), poly(phosphate-esters), polyurethanes, polyanhydrides, biomolecules, and blends thereof.

Bioabsorbable polyesters may be used and are described, e.g., in Pitt et al., “Biodegradable Drug Delivery Systems Based on Alipathic Polyesters: Application to Contraceptives and Narcotic Antagonists”, Controlled Release of Bioactive Materials, 19-44 Richard Baker ed., (1980). Aliphatic polyesters include homopolymers and copolymers of lactides (including lactic acid and D-, L-, and meso lactide), ε-caprolactone, glycolide (including glycolic acid and lactide/glycolide copolymers), hydroxybutyrate, hydroxyvalerate, dioxanone (e.g., para-dioxanone), trimethylene carbonate (and its alkyl derivatives), 1,4-dioxepan-2-one, 1,5-dioxepan-2-one, 6,6-dimethyl-1,4-dioxan-2-one, and polymer blends thereof. Bioabsorbable polyorthoesters may also be used and are described e.g., by Heller et al., “Release of Norethindrone from Poly(ortho Esters)”, Polymer Engineering Sci., 21:11, 727-31 (1981) and also by Heller in Handbook of Biodegradable Polymers, edited by Domb, Kost and Wisemen, Hardwood Academic Press (1997) p. 99-118. Polyorthoesters include, e.g., polyglycolic acid and polylactic acid such as poly-L-lactic acid (PLLA); poly D,L-lactic acid; and poly-D-lactic acid. Bioabsorbable polyphosphazenes are described, e.g., by Dunn et al., in U.S. Pat. Nos. 5,340,849; 5,324,519; 5,278,202; and 5,278,201. Polyphosphazenes, co-, ter- and higher order mixed monomer based polymers made from L-lactide, D,L-lactide, lactic acid, glycolide, glycolic acid, para-dioxanone, trimethylene carbonate and ε-caprolactone, are described by Allcock in The Encyclopedia of Polymer Science, Vol. 13, p. 31-41, Wiley Intersciences, John Wiley & Sons (1988) and by Vandorpe, Schacht, Dejardin and Lemmouchi in the Handbook of Biodegradable Polymers, edited by Domb, Kost and Wisemen, Hardwood Academic Press (1997), p. 161-182. Poly(amino acids) and pseudo-poly(amino acids) are described, e.g., by Pulapura et al., “Trends in the Development of Bioresorbable Polymers for Medical Applications,” J. of Biomaterials Appl., 6:1, 216-50 (1992); Poly(iminocarbonate) is described, e.g., in Kemnitzer and Kohn, Handbook of Biodegradable Polymers, edited by Domb, Kost and Wisemen, Hardwood Academic Press (1997), p. 251-272. Copoly(ether-esters) include, e.g., PEO/PLA and others described by Cohn and Younes, Journal of Biomaterials Research, Vol. 22 (1998), p. 993-1009, and by Cohn, Polymer Preprints (ACS Division of Polymer Chemistry) Vol. 30(1), (1989) p. 498. Polyalkylene oxalates include those described in U.S. Pat. Nos. 4,208,511; 4,141,087; 4,130,639; 4,140,678; 4,105,034; and 4,205,399. Polyanhydrides include those resulting from the polymerization of diacids of the form HOOC—C₆H₄—O—(CH₂)ₘ—O—C₆H₄—COOH where m is an integer from about 2 to about 8 and also include copolymers resulting from the copolymerization of these diacids with aliphatic alpha-omega diacids of up to 12 carbons. As is known in the art, the monomer ratios in polyanhydride copolymers may be varied so that the resulting copolymer is surface eroding. Polyoxaesters, polyoxaamides, and polyoxaesters containing amines and/or amido groups are described in one or more of U.S. Pat. Nos. 5,464,929; 5,595,751; 5,597,579; 5,607,687; 5,618,552; 5,620,698; 5,645,850; 5,648,088; 5,698,213 and 5,700,583. Bioabsorbable poly(phosphate-esters), e.g., poly(phosphates), poly(phosphonates) and poly(phosphites), are described, e.g., by Penczek et al., Handbook of Polymer Synthesis, Chapter 17: “Phosphorus-Containing Polymers”, p. 1077-1132 (Hans R. Kricheldorf ed., 1992) and in U.S. Pat. No. 6,153,212. Bioabsorbable polyurethanes are described, e.g., by Bruin et al., “Biodegradable Lysine Diisocyanate-based Poly-(Glycolide-co-ε-Caprolactone)-Urethane Network in Artificial Skin”, Biomaterials, 11:4, 291-95 (1990). Bioabsorbable polyanhydrides are described, e.g., by Leong et al., “Polyanhydrides for Controlled Release of Bioactive Agents”, Biomaterials, 7:5, 364-71 (1986).

Polymeric biomolecules may also advantageously be used with the rails 12, 12′, 120 and 312 or other portions of the stents according to the present invention. Polymeric biomolecules include naturally occurring materials that may be enzymatically degraded in the human body or those that are hydrolytically unstable in the human body. Such materials include albumin, alginate, gelatin, acacia, cellulose dextran, ficoll, hydroxyethyl cellulose, hydroxypropyl cellulose, hydroxypropylmethyl cellulose, carboxyethyl cellulose, carboxymethyl cellulose, fibrin, fibrinogen, collagen, elastin, dextran sulfate and absorbable biocompatable polysaccharides such as chitosan, deacetylated chitosan, starch, fatty acids (and esters thereof), glucoso-glycans and hyaluronic acid.

Other useful materials include bioabsorbable elastomers, preferably aliphatic polyester elastomers. In the proper proportions aliphatic polyester copolymers are elastomers. If used as coating materials, elastomers advantageously adhere well to the metal portions of the stent and can withstand significant deformation without cracking. Examples of suitable bioabsorbable elastomers are described in U.S. Pat. No. 5,468,253. Preferred bioabsorbable biocompatible elastomers are based on aliphatic polyesters, including elastomeric copolymers of ε-caprolactone and glycolide (preferably having a mole ratio of ε-caprolactone to glycolide from about 35:65 to about 65:35); elastomeric copolymers of ε-caprolactone and lactide, including L-lactide, D-lactide and blends thereof or lactic acid copolymers (preferably having a mole ratio of ε-caprolactone to lactide from about 35:65 to about 90:10); elastomeric copolymers of p-dioxanone (1,4-dioxan-2-one) and lactide including L-lactide, D-lactide and lactic acid (preferably having a mole ratio of p-dioxanone to lactide from about 40:60 to about 60:40); elastomeric copolymers of ε-caprolactone and p-dioxanone (preferably having a mole ratio of ε-caprolactone to p-dioxanone from about 30:70 to about 70:30); elastomeric copolymers of p-dioxanone and trimethylene carbonate (preferably having a mole ratio of p-dioxanone to trimethylene carbonate from about 30:70 to about 70:30); elastomeric copolymers of trimethylene carbonate and glycolide (preferably having a mole ratio of trimethylene carbonate to glycolide from about 30:70 to about 70:30); elastomeric copolymers of trimethylene carbonate and lactide including L-lactide, D-lactide, and blends thereof; or lactic acid copolymers (preferably having a mole ratio of trimethylene carbonate to lactide from about 30:70 to about 70:30) and blends thereof.

The present invention also includes introducing an agent into a body using one of the above-discussed stents. In a preferred embodiment, the agent(s) is carried by one or more of the rail elements 12, 12′, 120 and 312 and released within the body over a predetermined period of time. Local delivery of an agent is advantageous in that its effective local concentration is much higher when delivered by the stent than that normally achieved by systemic administration. The rail elements 12, 12′, 120 and 312, which are relatively inelastic in their transverse strength properties, may carry one or more of the above-referenced agents for applying to a vessel as the vessel moves into contact with the agent carrying rail element(s) 12, 12′, 120 and 312 after deployment of the stent within the vessel.

The above-discussed stents can deliver one or more known agents, including therapeutic and pharmaceutical agents, such as a drug, at a site of contact with a portion of the vasculature system or when released from a carrier as is known. These agents can include any known therapeutic drugs, antiplatelet agents, anticoagulant agents, antimicrobial agents, antimetabolic agents and proteins used for the treatment, prevention, diagnosis, cure, or mitigation of disease or illness; substances that affect the structure of function of the body; and prodrugs, which become biologically active or more active after placement in a given physiological environment. Agents may include medicaments, vitamins, mineral supplements. The agents may also include any of those disclosed in U.S. Pat. No. 6,153,252 to Hossainy et al. and U.S. Pat. No. 5,833,651 to Donovan et al., both of which are hereby incorporated by reference in their entirety.

Preferred agents usable with the rail elements disclosed herein are those that inhibit restenosis through any of a variety of approaches and include anti-inflammatory immuno-modulators including dexamethasone, m-prednisolone, interferon γ-lb, leflunomide, sirolimus, everolimus, tacrolimus, mycophenolic acid, mizoribine, cyclosporine, rapamycin, and tranilast; antiproliferatives including QP-2, taxol, actinomycine, methotrexate, angiopeptin, vincristine, mitomycin, statins, CMYC antisense, ABT-578, RestenASE, 2-chlorodeoxyadenosine, PCNA ribozyme, paclitaxel, rapamycin, everolimus and tacrolimus; migration inhbitors/ECM-modulators including batimastat, prolylhydroxylase inhibitors, halofuginone, C-proteinase inhibitors, probucol, rapamycin, everolimus and tacrolimus; and agents that promote healing and reendothelialization including BCP671, VEGF, and estrogen. Additional agents, such as those discussed below, can also be used.

Non-limiting examples of agents include those within the following therapeutic categories: analgesics, such as nonsteroidal anti-inflammatories (NSAIDs), steroidal anti-inflammatories, COX 2 selective inhibitors, opiate agonists and salicylates; angiogenesis inhibitors; antiasthmatics; antihistamines/antiprurities, such as H₁-blockers and H₂-blockers; anti-infectives, such as anthelmintics, anti-anaerobics, antibiotics, aminoglycoside antibiotics, antifungal antibiotics, macrolide antibiotics, miscellaneous β-lactam antibiotics, penicillin antibiotics, quinolone antibiotics, sulfonamide antibiotics, tetracycline antibiotics, antimicrobials, antibacterials, antimycobacterials, antituberculosis antimycobacterials, antiprotozoals, antimalarial antiprotozoals, antiviral agents, anti-retroviral agents, scabicides, and urinary anti-infectives; antiarthritics; antifibrinolytics; antineoplastics, such as alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics; calcium regulators; autonomic agents, such as anticholinergics, xanthines, mast cell stabilizers, antimuscarinic anticholinergics, ergot alkaloids, parasympathomimetics, cholinergic agonist parasympathomimetics, cholinesterase inhibitor parasympathomimetics, sympatholytics, α-blocker sympatholytics, β-blocker sympatholytics, sympathomimetics, and adrenergic agonist sympathomimetics; cardiovascular agents, such as antianginals, β-blocker antianginals, calcium-channel blocker antianginals, nitrate antianginals, antiarrhythmics, cardiac glycoside antiarrhythmics, class I, II, III, or IV antiarrhythmics, antihypertensive agents, α-blocker antihypertensives, angiotensin-converting enzyme inhibitor (ACE inhibitor) antihypertensives, β-blocker antihypertensives, calcium-channel blocker antihypertensives, central-acting adrenergic antihypertensives, diuretic anti-hypertensive agents, peripheral vasodilator anti-hypertensives, anti-lipidemics, inotropes, cardiac glycoside inotropes, and thrombolytics/fibrinolytics; dermatological agents, such as antihistamines, anti-inflammatory agents, corticosteroid anti-inflammatory agents, and antipruritics/local anesthetics; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as anti-diarrheals, antiemetics/antinauseants, gastrointestinal anti-inflammatory agents, salicylate gastrointestinal anti-inflammatory agents, anti-ulcer/anti-reflux agents, antacid anti-ulcer agents, gastric acid-pump inhibitor anti-ulcer agents, gastric mucosal anti-ulcer agents, H₂-blocker anti-ulcer agents, cholelitholytic agents, digestants, emetics, laxatives and stool softeners, and prokinetic agents; enzyme inhibitors; general anesthetics, such as halogenated anesthetics, barbiturate anesthetics, benzodiazepine anesthetics, and opiate agonist anesthetics; hematological agents, such as antianemia agents, hematopoietic antianemia agents, coagulation agents, anticoagulants, hemorheologic agents, hemostatic coagulation agents, antiplatelet agents, thrombolytic enzyme coagulation agents, and plasma volume expanders; hormones, hormone modifiers, and thyroid hormones, such as abortifacients, adrenal agents, adrenal corticosteroids, androgens, anti-androgens, antidiabetics, sulfonylurea antidiabetic agents, antihypoglycemic agents, progestins, estrogens, fertility agents, oxytocics, parathyroid agents, pituitary hormones, antithyroid agents, thyroid hormones, and tocolytics; immunobiologic agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, immunosuppressive anti-inflammatory agents, salicylate anti-inflammatory agents, skeletal muscle relaxants, neuromuscular blocker skeletal muscle relaxants, and reverse neuromuscular blocker skeletal muscle relaxants; anti-apoptotics; neurological agents, such as anticonvulsants, barbiturate anticonvulsants, benzo-diazepine anticonvulsants, anti-migraine agents, anti-parkinsonian agents, anti-vertigo agents, opiate agonists, and opiate antagonists; ophthalmic agents, such as anti-glaucoma agents, β-blocker anti-glaucoma agents, miotic anti-glaucoma agents, mydriatics, adrenergic agonist mydriatics, antimuscarinic mydriatics, ophthalmic anesthetics, ophthalmic anti-infectives, ophthalmic aminoglycoside anti-infectives, ophthalmic macrolide anti-infectives, ophthalmic quinolone anti-infectives, ophthalmic sulfonamide anti-infectives, ophthalmic tetracycline anti-infectives, ophthalmic agents, ophthalmic corticosteroid anti-inflammatory agents, and ophthalmic nonsteroidal anti-inflammatory drugs; psychotropic agents, such as antidepressants, heterocyclic anti-depressants, monoamine oxidase inhibitors (MAOIs), selective serotonin re-uptake inhibitors (SSRIs), tricyclic antidepressants, antimanics, antipsychotics, phenothiazine antipsychotics, anxiolytics, sedatives, and hypnotics, barbiturate sedatives and hypnotics, benzodiazepine anxiolytics, sedatives, and hypnotics, and psychostimulants; respiratory agents, such as antitussives, bronchodilators, adrenergic agonist bronchodilators, antimuscarinic bronchodilators, expectorants, mucolytic agents, respiratory anti-inflammatory agents, and respiratory corticosteroid anti-inflammatory agents; toxicology agents, such as antidotes, heavy metal antagonists/chelating agents, substance abuse agents, deterrent substance abuse agents, and withdrawal substance abuse agents; minerals; vitamins, such as vitamin A, vitamin B, vitamin C, vitamin D, vitamin E, and vitamin K; amino acids; and proteins, such as antibodies (e.g., monoclonal antibodies, polyclonal antibodies, and antibody fragments).

The following are examples of agents within the various therapeutic categories discussed above that can be used alone or with another one or more of these agents:

Analgesics include, e.g., para-aminophenol derivatives (e.g., acetaminophen), indole and indene acetic acids (e.g., etodalac), heteroaryl acetic acids (e.g., diclofenac and ketorolac), arylpropionic acids (e.g., ibuprofen), anthranilic acids (e.g., mefenamic acid and meclofenamic acid), enolic acids (e.g., tenoxicam and oxyphenthatrazone), nabumetone, gold compounds (e.g., gold sodium thiomalate), buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, methotrimeprazine, cinnamedrine hydrochloride, meprobamate, ketoprofen, flurbiprofen, naproxen, ramifenazone, meloxicam, fluazacort, celecoxib, rofecoxib, valdecoxib, nepafenac, ISV-205; angiogenesis inhibitors include, e.g., angiostatin (plasminogen fragment), vascular endothelial cell growth factor (VEGF), fibroblast growth factor (FGF), nitric oxide donors, antiangiogenic anithrombin III, cartilage-derived inhibitor (CD1), CD59 complement fragment, endostatin (collagen XVIII fragment), fibronectin fragment, gro-beta, heparinases, heparin hexasaccharide fragment, human chorionic gonadotropin (hCG), α-, β-, and γ-interferon, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF-4), prolactin 16 kD fragment, proliferin-related protein (PRP), retinoids, tetrahydrocortisol-S, thrombospondin-1 (TSP-1), transforming growth factor-beta (TGF-b), vasculostatin, vasostatin (calreticulin fragment), apolipoprotein E, TBC-2576; antiasthmatics include, e.g., ketotifen and traxanox; antidepressants include, e.g., nefopam, oxypertine, amoxapine, trazodone, maprotiline, phenelzine, desipramine, nortriptyline, tranylcypromine, fluoxetine, doxepin, imipramine, imipramine pamoate, isocarboxazid, trimipramine, and protriptyline; antidiabetics include, e.g., biguanides (e.g., metformin), sulfonylurea derivatives (e.g., tolbutamide, chlorpropamide, acetohexamide, tolazamide, and glimepiride), α-glucosidase inhibitors (e.g., acarbose), thiazolidinediones (e.g., troglitazone), and metglinide analogs (e.g., repaglinide); antihypertensive agents include, e.g., propanolol, propafenone, oxyprenolol, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine; antineoplastics include, e.g., cladribine (2-chlorodeoxyadenosine), nitrogen mustards (e.g., cyclophosphamide, mechlorethamine, melphalan, and chlorambucil), ethylenimines and methylmelamines (e.g., hexamethylmelamine and thiotepa), alkyl sulfonates (e.g., busulfan), nitrosoureas (e.g., streptozocin, carmustine (BCNU), methyl-CCNU and analogs), trazenes (e.g., dacarbazinine (DTIC)), platinum coordination complexes (e.g., carboplatin and cisplatin), procarbazine, hydroxyurea, mitotane, aminoglutethimide, camptothecin phenesterine, paclitaxel, docetaxel, vinca alkaloids (e.g., vinblastine, vincristine, and vinorelbine), epidipodophyllotoxins (e.g., etoposide (VP-16) and teniposide), tamoxifen, and piposulfan; anxiolytics include, e.g., lorazepam, buspirone, prazepam, chlordiazepoxide, oxazepam, clorazepate dipotassium, hydroxyzine pamoate, hydroxyzine hydrochloride, alprazolam, droperidol, halazepam, chlormezanone, and dantrolene; enzyme inhibitors include, e.g., selegiline or its hydrochloride salt, lazabemide, rasagiline, moclobemide, entacapone, tolcapone, nitecapone, Ro 40-7592, clozapine, risperidone, olanzapine, and quetiapine; immunosuppressives include, e.g., calcineurin inhibitors (e.g., cyclosporine and tacrolimus (FK-506)), antiproliferative/antimetabolic agents (e.g., sirolimus, QP-2, taxol, actinomycin, dactinomycin, daunorubicin, angiopeptin, mitomycine, bleomycin, doxorubicin, epirubicin, mitomycin, idarubicin, anthracyclines, mitoxantrone, plicamycin, CMYC antisense, ABT-578, RestenASE, 2-chloro deoxyadenosine, PCNA ribozyme, rapamycin, folic acid analogs (e.g., methotrexate), fluorouracil (5-FU), floxuridine, cytarabine, mercaptopurine, thioguanine, pentostatin, cyclophosphamide, thalidomide, chorambucil, leflunomide, batimastat, and mizoribine), everolimus, azathioprine, cytoxan, mycophenolic acid, mycophenolate mofetil, and tranilast; antimigraine agents include, e.g., ergotamine, isometheptene mucate, and dichloralphenazone; sedatives and hypnotics include, e.g., barbiturates (e.g., pentobarbital and secobarbital), flurazepam hydrochloride, triazolam, and midazolam; calcium-channel blocker antianginals include, e.g., nifedipine and diltiazem; nitrate antianginals include, e.g., nitroglycerin, isosorbide dinitrate, pentaerythritol tetranitrate, and erythrityl tetranitrate; antipsychotics include, e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine; antimanics include, e.g., lithium carbonate; antiarrhythmics include, e.g., bretylium tosylate, esmolol, verapamil, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine; antiarthritics include, e.g., phenylbutazone, sulindac, penicillanine, salsalate, piroxicam, indomethacin, meclofenamate, ketoprofen, auranofin, aurothioglucose, tolmetin, and tolmetin sodium; anti-gout agents include, e.g., colchicine and allopurinol; anticoagulants include e.g., danaparoid, lepirudin, dicumarol, acenocoumarol, heparin, heparin salts (e.g., heparin sodium), warfarin sodium, 4-hydroxycoumarin, phenprocoumon, indan-1,3 dione, anisindione, warfarin sodium, tissue factor pathway inhibitor (TFPI), tifacogin, ancrod, bromindione, clorindione, coumetarol, cyclocoumarol, 4-coumarinol, desirudin, dexran sodium sulfate, diphenadione, ethyl biscoumacetate, fluindione, hirudin, nadroparin calcium, nafamostat mesylate, oxazidione, phenindione, phosvitin, picotamide, sodium apolate, thrombocid, tioclomarol, warfarin, aprosulate sodium, ART 123, bivalirudin, BMS 189090, BMS 186282, BMS 189664, BMS 191032, corsevin M, CS 747, curdlan sulfate, DPC 423, DX 9065a, efegatran, fondaparinux sodium, GR 144053, inogatran, LB 30057, melagatran, MR 33, napsagatran, NSL 9403, SR 90107, YM 75466, ZK 805412, ZK 807834, OGS 15435, JTV 803, LY 287045, P 8720, RE 1492, Ro 43-8857, S 18326, S 31214, SK 549, SB 249417, SR 123781A, and UK 156406; thrombolytics/fibrinolytics include, e.g., urokinase, streptokinase, alteplase, phosphorylcholine, plasmin, plasminogen, angiokinase, anistreplase, prourokinase, reteplase, saruplase, tissue plasminogen activator, actinokinase, α2-antiplasmin, antithrombin, E 6010, fibrolase, lys-plasminogen, lanoteplase, lumbrokinase, metalloproteinase, monteplase, PAI proteinase inhibitor, pamiteplase, staphylokinase, and tenecteplase; antifibrinolytics include, e.g., aminocaproic acid; hemorheologic agents include, e.g., pentoxifylline; antiplatelet agents include, e.g., aspirin, ticlopidine, abciximab, clopidogrel, eptifibatide, tirofiban, and glycoprotein IIb/IIa inhibitors, argatroban, cilostazole, cloricromene, dalteparin, daltroban, defibrotide, dipyridamole, enoxaparin, iloprost, indobufen, isbogrel, lamifiban, lotrifiban nadroparin calcium, orbofiban, pamicogrel KBT 3022, plafibride, picotamide, ozagrel, ramatroban, reviparin sodium, ridogrel, roxifiban, satigrel, sibrafiban, sulotroban, taprostene, ticlopidine, triflusal, amrinone, cilostamide, dialzep, enoximone, milrinone, naftazone, pimilprost, pimobendan, sarpogrelate, sulfinpyrazone, vapiprost, vesnarinone, xemilofiban, zaprinast, zeria Z 335, A 02131-1, camonagrel, cangrelor, DMP 728, DMP 802, elarofiban, EMD 122347 FK 633, FXV 673, ifetroban, L 734217, lefradafiban,MK 852, ON 579, R 99224, RGD 039, RGD 891, RPR 109891, Ro 48-3657, Ro 44-3888, S 1197, SDZ-GPI 562, SL 650472, SM 20302, SR 121566A, SR 121787A, TA 993, TAK 029, XV 454, XV 459, YC-1, aspalatone, BAY 41-2272, BM 531, BM 14515, C 186-65, CS 570, FR 158999, fradafiban, L 750034, linotroban, ME 3277, MED 27, NQ 12, NQ 301, NQ 304, NSL 9511, NSP 513, 4-pentynoic acid, 3-[[4-[[4-(aminomethyl)-phenyl]amino]-1,4-dioxobutyl]-amino]-ethyl ester, RE 2047, SCH 79797, SM 10906, SR 25989, TP 9201, XJ 735, XR 300, XU 057, XU 063, XU 065, Y 909, ZD 2486, and ZD 9583; anti-apoptotics include, e.g., CGP 3466, CEP-1347/KT-7515, TCH-346, and WHI-P131; neurological agents include, e.g., timolol, dapiprazole, levobunolol, betaxolol, befunolol, carteolol, metipranolol, AMO-140, bunazosin, adaprolol, ISV-208, L-653328, cetamolol, H-216/44, KRG-332, levobetaxolol, metazosin, NCX-904, NCX-905, guanethidine, brimonidine, apraclonidine, AGN-195795, AGN-191103, AGN-190532, AGN-192172, AGN-193080, AGN-190837, talipexole, thiourea, dipivefrin, epinephrine, phenylephrine, cocaine, hydroxyamphetamine, naphazoline, tetrahydrozoline, levodopa, levodopa/carbidopa, levodopa/benserazide, amantadine, sumanirole, pergolide, pramipexole, ropinirole, bromocriptine, lisuride or 9, 10 dihydrolisuride, apomorphine or N-propylnoraporphine, N-propyl noraporphine, PHNO, N-0437 (racemate) and N-9023 (purified negative enantiomer), cabergoline, ciladopa, ABT-431, lergotrile, DIB1508Y, and ABT418m; selective serotonin re-uptake inhibitors (SSRIs) include, e.g., paroxetine, and serataline; anticonvulsants include, e.g., valproic acid, divalproex sodium, phenytoin, phenytoin sodium, clonazepam, primidone, phenobarbitol, carbamazepine, amobarbital sodium, methsuximide, metharbital, mephobarbital, mephenytoin, phensuximide, paramethadione, ethotoin, phenacemide, secobarbitol sodium, clorazepate dipotassium, and trimethadione; anti-parkinsonian agents include, e.g., ethosuximide; antihistamines/antipruritics include, e.g., hydroxyzine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, and methdilazine; calcium regulators include, e.g., calcitonin and parathyroid hormone; antibacterials include, e.g., amikacin sulfate, aztreonam, chloramphenicol, chloramphenicol palirtate, clindamycin, clindamycin palmitate, clindamycin phosphate, metronidazole, gentamicin sulfate, lincomycin hydrochloride, tobramycin sulfate, vancomycin hydrochloride, polymyxin B sulfate, colistimethate sodium, and colistin sulfate; antibiotics include, e.g., neomycin, streptomycin, chloramphenicol, cephalosporin, ampicillin, penicillin, tetracycline, and ciprofloxacin; antifungal antibiotics include, e.g., griseofulvin, ketoconazole, itraconizole, amphotericin B, nystatin, and candicidin; antiviral agents include, e.g., zidovudine (AZT), amantadine hydrochloride, ribavirin, and acyclovir; antimicrobials include, e.g., cephalosporins (e.g., cefazolin sodium, cephradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, cefadroxil, and cefuroxime sodium), penicillins (e.g., ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium), and erythromycins (e.g., erythromycin ethylsuccinate, erythromycin, erythromycin estolate, erythromycin lactobionate, erythromycin stearate, and erythromycin ethylsuccinate), and tetracyclines (e.g., tetracycline hydrochloride, doxycycline hyclate, minocycline hydrochloride, azithromycin, and clarithromycin); anti-infectives include, e.g., GM-CSF; sympathomimetics include, e.g., epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, epinephrine, and epinephrine bitartrate; anticholinergics include, e.g., ipratropium bromide, benzhexol, trihexphenidyl, benzotropine, diphenhydramine hydrochloride, orphenadrine, chlorphenoxamine, amitriptyline, doxepin, imipramine, nortriptyline, biperiden, ethopropazine, procyclidine, cycrimine, and ethopropzaine; xanthines include, e.g., aminophylline, dyphylline, metaproterenol sulfate, and aminophylline; mast cell stabilizers include, e.g., cromolyn sodium; bronchodilators include, e.g., salbutamol, budesonide, ketotifen, salmeterol, xinafoate, terbutaline sulfate, theophylline, nedocromil sodium, metaproterenol sulfate, flunisolide, and fluticasone proprionate; androgens include, e.g., danazol, testosterone cypionate, fluoxymesterone, ethyltestosterone, testosterone enathate, methyltestosterone; estrogens include, e.g., estradiol, estropipate, and conjugated estrogens; progestins include, e.g., methoxyprogesterone acetate, and norethindrone acetate; adrenal corticosteroids include, e.g., cortisol, cortisone, oxandrolone, creatine, erythropeotin, dehydroepiandrosterone triamcinolone, betamethasone, betamethasone sodium phosphate, dexamethasone, dexamethasone sodium phosphate, dexamethasone acetate, prednisone, prednisolone, methylprednisolone acetate suspension, triamcinolone acetonide, hydrocortisone sodium succinate, triamcinolone hexacetonide, hydrocortisone, hydrocortisone cypionate, fludrocortisone acetate, paramethasone acetate, prednisolone tebutate, and prednisolone acetate; thyroid hormones include, e.g., levothyroxine sodium; antihypoglycemic agents include, e.g., human insulin, purified beef insulin, purified pork insulin, glyburide, chlorpropamide, glipizide, tolbutamide, and tolazamide; anti-lipidemics include e.g., antiatherosclerotics and antihypercholesteremics (e.g., cholesteryl ester transfer protein (CETP) inhibitors, such as those disclosed in U.S. Pat. No. 6,458,850; ileal bile acid transport (IBAT) inhibitors, such as those disclosed in U.S. Pat. No. 6,458,851; and HMG CoA reductase inhibitors, such as those disclosed in U.S. Pat. No. 6,462,091), fibric acid derivatives (e.g., clofibrate, fenofibrate, ciprofibrate, benzafibrate, clinofibrate, binifibrate and gemfibrozil), and nicotinic acid derivatives (e.g., nicotinic acid, niceritrol, and acipimox), dextrothyroxine sodium, probucol, pravastatin, atorvastatin, lovastatin, and niacin; antiulcer/antireflux agents include, e.g., famotidine, cimetidine, and ranitidine hydrochloride; antiemetics/antinauseants include, e.g., meclizine hydrochloride, nabilone, prochlorperazine, dimenhydrinate, promethazine hydrochloride, thiethylperazine, and scopolamine; collagen synthesis inhibitors include, e.g., prolyl hydroxylase inhibitors, C-proteinase inhibitors, and halofuginone; vitamins include oil-soluble vitamins (e.g., vitamins A, D, E, and K); amino acids include, e.g., valine, leucine, and isoleucine; proteins include, e.g., cyclophilin, antithymocyte globulin, immunoglobulin, muromonab-CD3, daclizumab, basiliximab, infliximab, etanercept, DNase, alginase, L-asparaginase, superoxide dismutase (SOD), lipase, metallothionine, a polipoprotein E, oxandrolone, creatine, dehydro epiandrosterone, platelet derived growth factor, fibrin, fibrinogen, collagen, interleukins 1 through 18, luteinizing hormone releasing hormone (LHRH), gonadotropin releasing hormone (GnRH), and transforming growth factor-β (TGF-β), tumor necrosis factor-α and β (TNF-α and β), nerve growth factor (NGF), growth hormone releasing factor (GHRF), epidermal growth factor (EGF), fibroblast growth factor homologous factor (FGFHF); hepatocyte growth factor (HGF); insulin growth factor (IGF), invasion inhibiting factor-2 (IIF-2), bone morphogenetic proteins 1-7 (BMP 1-7), somatostatin; thymosin-α-1, and γ-globulin. Various biologically active forms of these proteins, including recombinant forms, mutants, complements, analogs, derivatives, and fragments are also contemplated. Other useful agents include nucleic acids (e.g., sense or anti-sense nucleic acids encoding any therapeutically useful protein, including any of the proteins described herein).

A description of other categories of useful agents and other individual agents can be found in Martindale, The Extra Pharmacopoeia, 30^{th} Ed. (The Pharmaceutical Press, London 1993).

Examples of other agents that may be delivered using the stent of the present invention include chlorhexidine, estradiol cypionate, estradiol valerate, flurbiprofen sodium, ivermectin, nafarelin, beta-glucan, bovine immunoglobulin, bovine superoxide dismutase, HIV-1 immunogen, human anti-TAC antibody, CD34 antibody, recombinant human growth hormone (r-hGH), recombinant human hemoglobin (r-Hb), recombinant human mecasermin (r-IGF-1), lenograstim (G-CSF), recombinant thyroid stimulating hormone (r-TSH), topotecan, aldesleukin, atenolol, epoetin alfa, leuprolide acetate, ceftriaxone, ceftazidime, oxaprozin, breveldin, valacyclovir, urofollitropin, famciclovir, flutamide, enalapril, mefformin, itraconazole, gabapentin, fosinopril, tramadol, lorazepan, follitropin, omeprazole, fluoxetine, lisinopril, tramsdol, levofloxacin, zafirlukast, growth hormone, granulocyte stimulating factor, nizatidine, bupropion, perindopril, erbumine, adenosine, alendronate, alprostadil, benazepril, bleomycin sulfate, dexfenfluramine, fentanyl, flecainid, gemcitabine, glatiramer acetate, granisetron, lamivudine, mangafodipir trisodium, mesalamine, metoprolol fumarate, miglitol, moexipril, monteleukast, octreotide acetate, olopatadine, paricalcitol, somatropin, sumatriptan succinate, tacrine, trovafloxacin, dolasetron, finasteride, isradipine, lansoprazole, terbinafine, pamidronate, didanosine, cisapride, venlafaxine, fluvastatin, losartan, imiglucerase, donepezil, valsartan, fexofenadine, BCP 671, adapalene, doxazosin mesylate, mometasone furoate, ursodiol, enalapril maleate, felodipine, nefazodone hydrochloride, valrubicin, albendazole, conjugated estrogens, medroxyprogesterone acetate, nicardipine hydrochloride, zolpidem tartrate, amlodipine besylate, ethinyl estradiol, rubitecan, amlodipine besylate/benazepril hydrochloride, etodolac, paroxetine hydrochloride, atovaquone, podofilox, betamethasone dipropionate, pramipexole dihydrochloride, Vitamin D₃ and related analogs, quetiapine fumarate, candesartan, cilexetil, fluconazole, ritonavir, flumazenil, carbemazepine, carbidopa, ganciclovir, saquinavir, amprenavir, sertraline hydrochloride, carvedilol, halobetasolproprionate, sildenafil citrate, chlorthalidone, imiquimod, simvastatin, citalopram, irinotecan hydrochloride, sparfloxacin, efavirenz, cisapride monohydrate, tamsulosin hydrochloride, mofafinil, letrozole, terbinafine hydrochloride, rosiglitazone maleate, diclofenac sodium, lomefloxacin hydrochloride, tirofiban hydrochloride, telmisartan, diazapam, loratadine, toremifene citrate, dinoprostone, mefloquine hydrochloride, trandolapril, tretinoin, nelfinavir mesylate, indinavir, beclomethasone dipropionate, isotretinoin, tamoxifen citrate, nimodipine, latanoprost, travoprost, unoprostone, AL-10682, AL-3138, AGN-191976, PhXA-34, AL-16082, bimatoprost, ethanolamide, dorzolamide, brinzolamide, acetazolamide, methazolamide, L-662583, MK-927, L-693612, L-685393, mannitol, glycerol, isosorbide, physostigamine, echothiophate, acetylcholine, methacholine, pilocarpine, aceclidine, carbachol, demecarium, isoflurophate, memantine, iomerizine, H-7, SR-43845, enalkiren, Y-39983, GPI-5693, anadamide, L-768242, L-759787, dexanabinol, collagenase ABC, iomefloxacin, iosartan, CS-088, mecobalamin, ISV-900, cardiotrophin-1, S-1033, D-22A, pentigetide, lerdelimumab, DE-085, SR-121463, org-34517, octamer, NNC-26-9100, KSR-592, A-75169, ethacrynate sodium, SDZ-GLC-756, rostaporfin, proxodolol, WIN-552122, OSA-8302, AL-16049, naboctate, L-696986, AL-4333A, vaninolol, PCA-50941, HGP-32, AGN-192836, AGN-191970, WP-934, ACC-9002, AL-4623A, L-4414A, CK-119, alprenoxime, CBT-101, AGN-191151, H 21644, SL 1111, GPI-5232, eliprodil, tilisolol, lomerizine, riluzole, lamotrigine, dextromethorphan, EAAT2, topiramate, AP5, CPP, selfotel or CGS 19755, CGP 37849, CGP 39551, CGP 40116, NPC 17742, aptiganel/CNS 1102, dextromethorphan and enzyme inhibitor, FR 115427, ketamine, ketobemidone, methadone, dizocilpine or MK 801, PCP, pethidine, RPR-1 19990, LY-300164 or talampanel, CNQX, DNQX, LY 215490, NNC 079202 or NBQX, NS 257, GYKI 52466, cyclothiazide, IDRA 21, DCG-IV, glycine, AP4, t-ACPD, L-SOP, L-AP3, S-4C3HPG, S-4CPG, MAP-4, RS-M4CPG, N-(3-[5-chloro-1-(4-chlorophenyl[indan-1-yl]propyl)-N-methylalanine, SR-57746A, T-588, 3,4 diaminopyridine, CPC-304, CPC-317, PD-176078, cephalosporin ceftriaxone, huperzine A, 10-methylhuperzine A, 10,10 dimethyl huperzine A, huperzine B, nicotine, epibaticline, cytosine, lobeline, anabasine, CNTF, BNDF, rhIGF-1, myotrophin mecasermin, Somatomedin C, GDNF, liatermin, neurturin, PEDF, FKBO-neuroimmunophilin ligands, AIT-082, leteprinim potassium, neotrofinT, emfilermin, CT-1, NT-3, NT-4/5, EHT 201, EHT 202, genistein, RX-77368, MK-771, JTP-2942, GPI-5000, ZVAD fink, 3-(2-phenyl-2-oxoethyl)-4,5-dimethylthiazolium salt, nordihydroguaiaretic acid, L-655238, Bay-X-1005, ML-3000, zileuton, oxothiazolidine carboxylate, ARR 17477, SOD, recombinant human CuZn-SOD, glutathione, glutathione peroxidase, catalase, nitric oxide synthase, vitamin E, vitamin C, selenium, acetylcysteine, seleginine, pycnogenol, co-enzyme Q10, beta carotene, PC 01, SC-55858, edaravone, iron (III) porphyrins, chromomycin, daunomycin, olivomycin, WP-631, DHEA, baclofen, tizandidine, dronabinol, diazepam, AVP-923, amitriptylene, fluvoxamine, sertraline, glycopyrrolate, copolamine, trihexyphenidyl, clonidine, propantheline, tropine, docusate sodium, tolterodine, TA-0910, ubiquinone, alpha lipoic acid, NAC, polyphenols, pregnenolone, threonine, methylcobalamin, metaxalone, tizanadine, carisoprodol, cyclobenzaprine, tramadol, potassium, calcium, zinc, magnesium, botulinum neurotoxin, succinylcholine, decamethonium, quinine, tetrahydrocannabinol, d-tubocurarine, atracurium, doxacurium, mivacurium, cistracurium besilate, pancuronium, pipecuronium bromide, rapacuronium bromide, rocuronium, vecuronium bromide, atracurium, suxamethonium; alcuronium, curare, metocurine, gallamine, nitrazepam, nordazepam, vigabatrin, procaine, chloroquine, gluthathione, odansetron, memantadine, GPI-1046, eradoline U-69 593, KW 6002, remacemide, dextromethorphan, NS-2214, CD133 antigen, CD34 antigen and reboxetine.

In addition to the above agents, there are a number of viruses, live or inactivate, including recombinant viruses that may, with the stent of the present invention, be used to deliver nucleic acids to the vessel walls of a lumen. Disorders that can be treated using viral delivery are described in U.S. Pat. No. 5,833,651. Examples of disorders that may be treated in this manner include, e.g., cell proliferation resulting from stenosis (for example using suicide genes or targeting cell-cycle regulatory genes); damage associated with myocardial infarction or aneurysms (targeting fibroblast growth factor or transforming growth factor β and protease respectively); atherosclerosis (for example, targeting high density lipoprotein); familial hypercholesterolemia (targeting the low density lipoprotein receptor), hypercoagulable states (targeting tissue-plasminogen activator), refractory diabetes mellitus (for example, targeting insulin) as well as diseases not necessarily associated with the vasculature, including, but not limited to, muscular dystrophy, cystic fibrosis, digestive disorders, cancer, inherited disease, colitis, benign prostatic hypertrophy, transplant rejection or transplant vasculopathy (targeting for example, leukocyte adhesion molecule or cytokines respectively), and the like. Treatment involves either the expression of a gene to provide a therapeutic effect to a cell or the expression of a gene to i) replace a mutated gene in a cell, ii) augment expression of a protein in a cell, or iii) inhibit a gene in a cell.

Of the therapeutic categories specified above, one set of preferred categories are those associated with treating vascular conditions that may or are likely to require a stent. Other preferred categories are those associated with the prevention or treatment of restenosis or side effects (e.g., infection) possibly accompanying stent insertion. Preferred therapeutic categories include hematological agents, preferably antiplatelet agents and anticoagulants; anti-infectives, preferably antimicrobials, antibacterials, antiviral agents, and antibiotics; immunobiologic agents, preferably immunosuppressives; proteins, preferably antibodies; cardiovascular agents, preferably anti-lipidemics, and thrombolytics/fibrinolytics; angiogenesis inhibitors; anti-apoptotics; antineoplastics; and collagen synthesis inhibitors.

The above agents may be used in any known pharmaceutically acceptable form. The term “pharmaceutically acceptable” refers to the agents being appropriate for use in vivo. For example, pharmaceutically acceptable forms include various metallic ion and organic ion forms. Metallic ions include, but are not limited to, alkali metal ions, alkaline earth metal ions and other physiological acceptable metal ions. Exemplary ions include aluminum, calcium, lithium, magnesium, potassium, sodium and zinc ion forms, where the ions are in their usual valences. Preferred organic ions include protonated tertiary amines and quatenary ammonium cations, including in part, trimethylamine, diethylamine, N,N′-dibenzylethylenediamine, chloroprocaine, choline, diethanolamine, ethylenediamine, meglumine (N-methylglucamine) and procaine.

Also included as pharmaceutically acceptable forms are various acid forms of the above agents. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, and benzoic acid. Further pharmaceutically acceptable forms include various salt forms of the above agents. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Other pharmaceutically acceptable salt forms are the base addition salt forms of the agents described above. Illustrative pharmaceutically acceptable base addition salts include metallic ion salts and organic ion salts. Preferred metallic ion salts include appropriate alkali metal (group Ia) salts, alkaline earth metal (group IIa) salts and other known physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine.

Also, other pharmaceutically acceptable forms of the above agents include the various isomeric forms (e.g., purified structural isomers; purified stereoisomers such as diastereomers and enantiomers; and purified racemates), tautomers, esters, amides and prodrugs of these agents.

These agents can be applied using a known method such as dipping, spraying, impregnation or any other technique described in the above-mentioned patents that have been incorporated by reference. Applying the agents to the rail elements 12, 12′, 120 and 312 avoids the mechanical disruption that occurs when coated elastic support elements are expanded. In this manner drug coatings applied to the stent rail elements 12, 12′, 120 and 312 may be used with support elements formed of materials that are otherwise unsuitable for coating.

Any one or more of the above-discussed agents may be coated onto the rails 12, 12′, 120 and 312, and other parts if desired, of the stent in any conventional manner, such by a spray coating, vapor deposition, simple dip coating or, if a thicker coating of the therapeutic agent is desired, multiple dip coatings of the same or multiple agents. The agents may be applied directly onto the rails 12, 12′, 120 and 312 in multiple layers, in grooves formed into an outer surface of these rails using a conventional technique, such as molding, laser etching/cutting or chemical etching/cutting, recesses (inlays) formed in the outer surface of these rails or in openings formed through these rails by any of the above-mentioned techniques.

Methods for spray coating a stent are described, e.g., in U.S. Pat. Nos. 5,464,650 and 5,833,651. Alternatively, a thin film of a therapeutic agent may be molded over the stent framework, as described in U.S. Pat. No. 4,866,062. A dditionally, rapamyacin analogs could be used on the rails to provide additional surface area for dry delivery of agents.

In general, multiple dipping involves applying several thin layers of the agent, while in liquid form (e.g., a solution, dispersion, or emulsion) of appropriate viscosity, and allowing each liquid layer to dry between successive applications. Drying may be carried out simply by evaporation in air or promoted by heating, including baking or heat flashing, or even osmotic moisture removal, for example, by using a semipermeable membrane. Otherwise, the formation of a solid, adhering layer may be accomplished through chemical or biological transformations occurring on the stent surface as described, for example in U.S. Pat. No. 4,548,736 where fibrin is solidified onto the stent by carrying out the clotting reaction between fibrinogen and thrombin.

Active flow systems are also possible. For example, U.S. Pat. No. 6,153,252 describes a method using fluid flow or movement through the passages in a perforated medical device to avoid the formation of blockages or bridges. The fluid flow can be created by using a perforated manifold inserted in the stent to circulate the coating fluid through the passages or by placing the stent on a mandrel or in a small tube that is moved relative to the stent during the coating process.

Another possibility for incorporation of a therapeutic agent is through the use of an active material that promotes physical or chemical adsorption. As described in WO 01/68158, an activated form of carbon known as a fullerene can promote the chemical binding of various biological agents (e.g., antibodies) to the surface of the rails 12, 12′, 120 and 312 for therapeutic delivery. In the same manner, various stent materials described previously (e.g., polymeric materials) may be chemically modified, such as by the incorporation of a co-monomer, to introduce functional groups that chemically interact or bind to a given therapeutic agent.

The rail elements 12, 12′, 120 and 312 can also be coated with a smooth nanoporous ceramic layer of aluminum oxide available from AlCove GmbH of Germany. This coating is suitable for releasing an agent such as those discussed above while provide the stent with high stability and flexibility. Such a coating can be used in place of a coating of one the discussed polymers.

The use of agent carrying rail elements 12, 12′, 120 and 312 can reduce the complexity and cost of manufacturing agent carrying stents because the rail elements 12, 12′, 120 and 312, which are uniform symmetrical elements, can be fabricated in a bulk process and, for example, ribbon coated with one or more agents, including therapeutic drugs, and spooled. The rail elements 12, 12′, 120 and 312 can be fabricated in forms that allow higher levels of drug loading and controlled dry release than conventional drug delivery stents. The individual agent carrying rail elements 12, 12′, 120 and 312 can be cut to size from a long ribbon of material and introduced through the radial support elements to form a stent according to the present invention.

Additionally, multiple rail elements cut from different ribbons and carrying the same or different agents can be used in the same stent. For example, if the stent includes three rails elements, the first rail element can carry one agent, the second rail element can carry a second agent that is different from the first agent and the third rail can carry a third agent. The third agent can be the same as one of the agents carried by the other two rail elements or different from the agents carried by the other two rail elements. As a result, the stents according to the present invention permit customization of the agents delivered to the body by allowing different rail elements carrying the same or different agents to be introduced through the support elements along the length of a single stent. Additional customizing of a stent can be achieved using rail elements that include different longitudinal sections carrying different agents.

In yet another embodiment, each of the rail elements contains more than one agent that may be released either simultaneously, at completely different times or delivery may overlap in time. The release rates of the individual agents or of all agents can be customized for a particular patient or condition using biocompatible polymers and manufacturing methods described above. This would allow the delivery of drug to be optimized to the normal healing processes with the appropriate drug at the right concentration delivered at the desired point in time.

The agents applied in separate layers can be the same agent, different agents with different time releases or different agents intended to be released simultaneously or in successive order. In either instance, barrier layers can cover the different layers of agents. For example, a first barrier layer could cover the rail surface, a first drug layer could be applied on top of the barrier layer and a separation layer applied over the first drug layer. A second drug layer could be applied over the separation layer and then a cover layer could be applied over the second drug layer. More than two drug layers can be applied to the rails. The cover and separation layers can be chosen to provide predetermined and independent time release of the applied agents that they cover.

The different agents discussed above can be applied on different rails or different portions of the same rail. As a result, numerous combinations of agents can be applied to the rails. For example, each complete rail or coated portion of a rail can include one or more layers of the same or different agents. Hence, one rail could be coated with different agent combinations at different locations along its length.

In an alternative embodiment, both the rail elements and the support elements of a single stent carry one or more of the above-discussed agents. The agent(s) carried by the hoops can be the same as, or different from, the agents carried by the rail elements. Additionally, the agent(s) carried by one or more of the rail elements can be carried by some of the support elements, while the remaining support elements and rail elements can carry the same or different agents.

It is contemplated that the various elements of the present invention can be combined with each other to provide the desired flexibility. For example, support element designs can be altered and various support element designs combined into a single stent with/without any one of the above-discussed rails. Similarly, the number, shape, composition and spacing of the rail elements can be altered to provide the stent with different properties. Additionally, the device can have varying numbers and placement of the bridge elements. The properties of any individual stent would be a function of the design, composition and spacing of the hoops, rails and bridges.

Thus, while there have been shown and described and pointed out fundamental novel features of the present invention as applied to preferred embodiments thereof, it will be understood that various omissions and substitutions and changes in the form and details of the devices illustrated, and in their operation, and in the method illustrated and described, may be made by those skilled in the art without departing from the spirit of the invention as broadly disclosed herein.

All of the above-discussed patents and publications are hereby expressly incorporated by reference as if they were written directly herein.

1. A stent for introducing within a body comprising:
a plurality of vessel support elements, a first one of said vessel support elements forming a first end support element and second one of said support elements forming a second end support element; and
at least one support rail element extending between said end support elements and including a curved end section for extending beyond one of the end support elements, wherein a plurality of said vessel support elements are moveable along and relative to said at least one support rail element.

2. The stent according to claim 1, wherein said at least one support rail element includes a plurality of curved end sections.

3. The stent according to claim 2, wherein said at least one support rail element includes a plurality of elongated sections extending between the curved end sections located at opposite ends of said stent.

4. The stent according to claim 3, wherein said elongated sections are integrally connected to each other by respective curved end sections.

5. The stent according to claim 4, wherein said stent includes a single support rail element that extends along multiple axes of said stent, said axes being substantially parallel to the longitudinal axis of the stent.

6. The stent according to claim 2, wherein said at least one support rail element includes a single elongated member extending between respective curved end sections, and wherein said respective curved end sections have terminal ends secured to a respective one of said vessel support elements.

7. The stent according to claim 1, wherein said at least one support rail element comprises a plurality of elongated sections and a curved section extending between said elongated sections.

8. The stent according to claim 7, wherein said elongated sections each have a first end secured to the first end support element and a second end integral with said curved section.

9. The stent according to claim 8, wherein said second ends of said elongated sections are free of a direct connection to said second end support element.

10. The stent according to claim 1, wherein said at least one support rail element comprises a support rail element that has a terminus secured to a vessel support element positioned between said first end support element and said second end support element.

11. The stent according to claim 1, wherein at least one of said vessel support elements or at least one of said support rail elements comprising a biocompatible material.

12. The stent according to claim 1, wherein at least one of said vessel support elements or at least one of said support rail elements comprising a base material having a biocompatible covering.

13. The stent according to claim 1, wherein at least one of said vessel support elements or at least one of said support rail elements comprising at least one agent for delivery to the body.

14. The stent according to claim 1, wherein at least one of said vessel support elements or at least one of said support rail elements comprising a base material having a coating of at least one agent for delivery to the body.
